(19) United States
(12) Patent Application Publication
(10) Pub. No.: US 2005/0233991 A1
(43) Pub. Date: Oct. 20, 2005

(54) Methods and compositions for the identification, assessment, and therapy of small cell lung cancer

(76) Inventor: Ravi Salgia, Park Ridge (US)

Correspondence Address: Novartis;Corporate Intellectual Property, One Health Plaza, Building 430, East Hanover, NJ, 07936-1080, US

(21) Appl. No.: 10/507,549

(22) PCT Filed: Mar. 20, 2003
(86) PCT No.: PCT/EP03/02916

§ 371 Date: Mar. 3, 2005

Related U.S. Application Data
(60) Provisional application No. 60/366,370, filed on Mar. 20, 2002.

(51) Int. Cl. A61K 48/00; A61K 38/54

(52) U.S. Cl. 514/44; 514/2

(57) ABSTRACT

The invention provides novel methods and compositions for modulating small cell lung cancer (SCLC) proliferation and metastasis through modulation of CXCR4 activity or expression. Also provided are methods for identifying compounds that modulate SCLC proliferation and metastasis through modulation of CXCR4 activity or expression. Further provided are methods for treating SCLC proliferation and metastasis, as well as methods for determining whether subjects are suitable candidates for treatment of SCLC via modulation of CXCR4 activity.

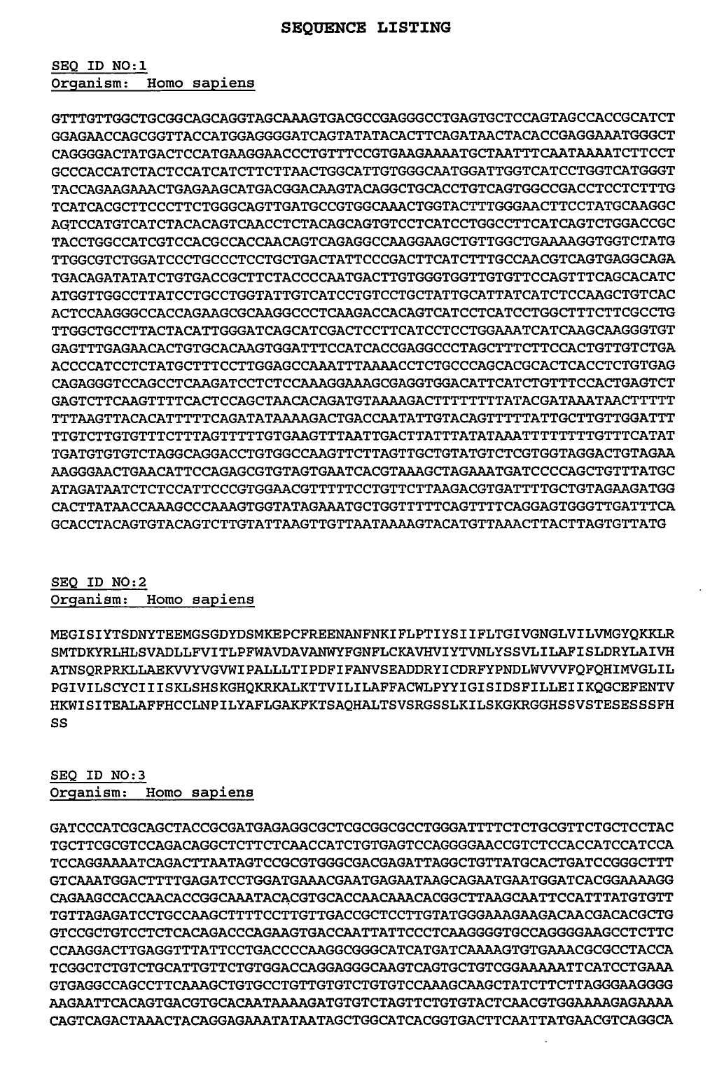

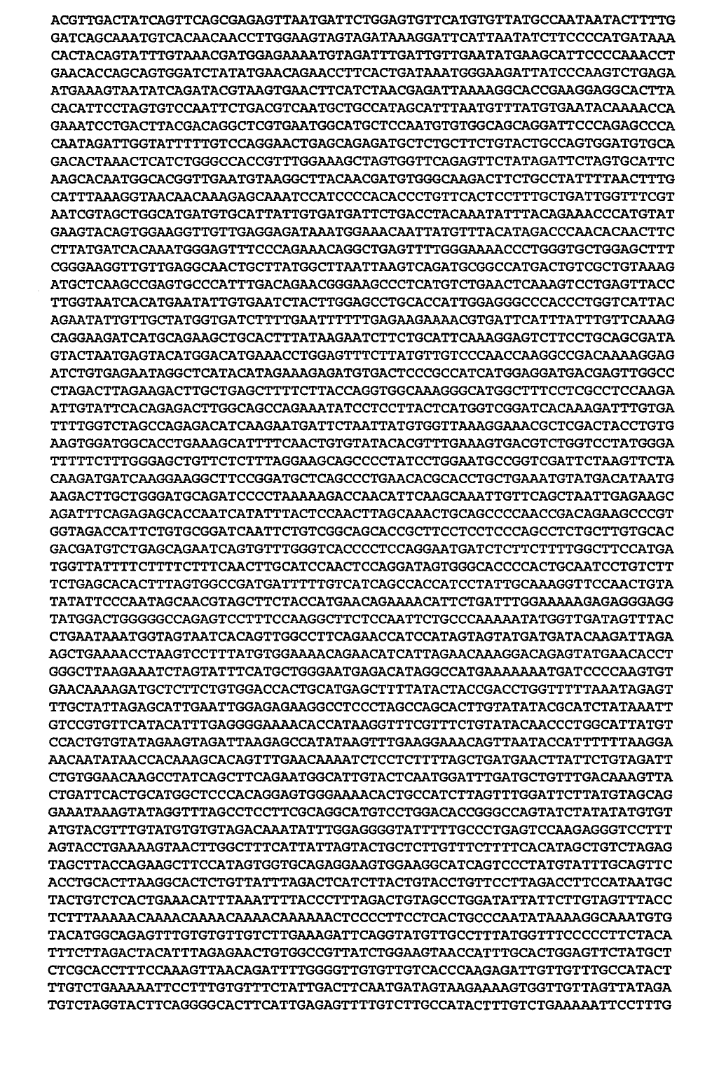

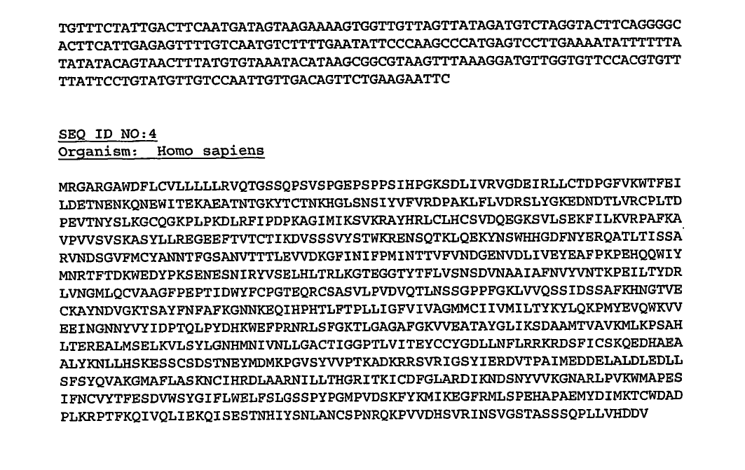

### BACKGROUND OF THE INVENTION

Small cell lung cancer (SCLC) is an aggressive cancer with characteristic early metastasis. Even with the best available therapy, the overall survival for patients with SCLC is only 12% for early disease and 2% for metastatic disease. Metastatic disease is the usual presentation for approximately two thirds of the patients with SCLC. Usual sites of metastasis include the liver, the adrenal glands, the brain, the bone, and the bone marrow (Chute, J. P. et al. (1999) J. Clin. Oncol. 17:1794-801; Chute, J. P. et al. (1997) Mayo Clin. Proc. 72:901-12; Salgia, R. (1996) Adv. Oncol. 12:8-15). Understanding the mechanisms of metastasis in SCLC to the lymph nodes and various organs is crucial in designing future therapeutics.

SCLC is characterized by overexpression of several receptor tyrosine kinases (RTKs). Some of these RTKs are proto-oncogenes and key regulators of cell growth, differentiation, survival, and motility. Recent work has begun to identify novel therapeutic agents for SCLC that target these RTKs (Gibbs, J. B. and Oliff, A. (1994) Cell 79:193-8; Levitzki, A. and Gazit, A. (1995) Science 267:1782-8). The c-Met and c-Kit RTKs have been identified as being important in SCLC. The c-Kit receptor, a proto-oncogene product with a molecular weight of 145 kDa, is a class III RTK similar to c-Fms, Flt3, and platelet derived growth factor receptor (PDGFR). The c-Kit protein contains five immunoglobulin-like domains in the extracellular regions, a transmembrane domain, and a cytoplasmic domain with two kinase domains separated by a kinase insert (Ashman, L. K. (1999) Int. J. Biochem. Cell Biol. 31:1037-51; Linnekin, D. (1999) Int. J. Biochem. Cell Biol. 31:1053-74; Matthews, W. et al., (1991) Cell 65:1143-52). Approximately 70% of SCLC tumor specimens and cell lines coexpress c-Kit and its natural ligand, stem cell factor (SCF). The SCF/c-Kit pathway is functional in an autocrine or a paracrine fashion in SCLC (Hibi, K. et al. (1991) Oncogene 6:2291-6; Krystal, G. W. et al. (1996) Cancer Res. 56:370-6; Plummer, H., 3^{rd} et al. (1993) Cancer Res. 53:4337-42; Rygaard, K. et al. 91993) Br. J. Cancer 67:37-46; Sekido, Y. et al. (1991) Cancer Res. 51:2416-9; Sekido, Y. et al. (1993) Cancer Res. 53:1709-14; Yamanishi, Y. et al. (1996) Jpn. J. Cancer Res. 87:53442. The c-Kit receptor can be inhibited by a variety of inhibitors, including the novel tyrosine kinase inhibitor imatinib mesylate (also referred to as STI571 or Gleevec™ (Krystal, G. W. et al. (1997) Cancer Res. 57:2203-8; Krystal, G. W. et al. (2001) Cancer Res. 61:3660-8; Krystal, G. W. et al. (2000) Clin. Cancer Res. 6:3319-26; Tuveson, D. A et al. (2001) Oncogene 20:5054-8; Wang, W. L. et al. (2000) Oncogene 3521-8)). STI571, having the chemical name 4-(4-methylpiperazin-1-ylmethyl)-N-[4-methyl-3-(4-pyridin-3-yl)-pyrimidin-2-ylamino)-phenyl]-benzamide, is further described in EP 0 564 409 and, in the form of the methane sulfonate salt, especially in the preferred β-crystal form, in WO 99/03854.

In hematopoietic cells, the c-Kit receptor has been shown to functionally interact with a variety of molecules, including chemokine receptors (Dutt, P. et al. (1998) J. Immunol. 161:3652-8; Kim, C. H. and Broxmeyer, H. E. (1998) Blood 91:100-10; Lataillade, J. J. et al. (2000) Blood 95:756-68; Peled, A. et al. (1999) Science 283:845-8) (13, 21, 26, 33). Given the lack of existing therapies to treat SCLC, particularly metastatic SCLC, there exists a need to identify additional molecular targets that may be used in SCLC for the identification of novel therapies.

### SUMMARY OF THE INVENTION

The present invention provides methods and compositions for the identification, assessment, and therapy of small cell lung cancer (SCLC). The present invention is based, at least in part, on the discovery that CXCR4 is ubiquitously expressed, and c-Kit is variably expressed, in SCLC cells. The present invention is further based on the discovery that treatment of SCLC cells with stromal cell derived factor-1α (SDF-1α) and/or stem cell factor (SCF) (the ligands for CXCR4 and c-Kit, respectively) induces proliferation and regulates adhesion, motility, and cell shape of SCLC cells. The present invention is still further based on the discovery that PI3-Kinase (PI3-K) regulates SDF-1α induced cell motility of SCLC cells, that CXCR4- and c-Kit cooperatively induce morphological changes in SCLC cells, that SDF-1α and SCF independently regulate phosphorylation of Akt and p70 S6 kinase, and that STI571 (a c-Kit inhibitor also referred to as imatinib mesylate or Gleevec™) and LY294002 (a PI3-K inhibitor) inhibit signal transduction of the CXCR4 and c-Kit pathways.

Accordingly, in one embodiment, the invention provides methods of inhibiting cellular proliferation, cellular movement or motility, and morphological change in a small cell lung cancer cell population, comprising contacting the population with a CXCR4 inhibitor. In another embodiment the invention provides methods of modulating cellular adhesion in a small cell lung cancer cell population comprising contacting the population with a CXCR4 inhibitor. In a preferred embodiment, PI3-K activity in the small cell lung cancer cell population is down-regulated.

In one embodiment, the CXCR4 inhibitor binds to CXCR4 or to SDF-1α. In another embodiment, the CXCR4 inhibitor is an antibody or antibody fragment. In still another embodiment, the CXCR4 inhibitor is a small molecule, for example, AMD-3100, ALX40-4C, T22, T140, Met-SDF-1beta, T134, or AMD-3465. In another embodiment, the methods of the invention further comprise contacting the small cell lung cancer cell population with a receptor tyrosine kinase inhibitor, for example, a c-Kit inhibitor such as imatinib mesylate (Gleevec™).

In another embodiment, the invention provides methods of treating a subject with small cell lung cancer comprising administering a CXCR4 inhibitor to the subject. In one embodiment, the CXCR4 inhibitor binds to CXCR4 or to SDF-1α. In another embodiment, the CXCR4 inhibitor is an antibody or antibody fragment. In still another embodiment, the CXCR4 inhibitor is a small molecule, for example, AMD-3100, ALX40-4C, T22, T140, Met-SDF-1beta, T134, or AMD-3465. In another embodiment, the methods further comprise administering a receptor tyrosine kinase inhibitor, for example, a c-Kit inhibitor such as imatinib mesylate (Gleevec™), to the subject. In a preferred embodiment, PI3-K activity in the small cell lung cancer cells of the subject is down-regulated.

In another embodiment, the invention provides methods of inhibiting metastasis of small cell lung cancer in a subject comprising administering a CXCR4 inhibitor to the subject. In one embodiment, the CXCR4 inhibitor binds to CXCR4 or to SDF-1α. In another embodiment, the CXCR4 inhibitor is an antibody or antibody fragment. In still another embodiment, the CXCR4 inhibitor is a small molecule, for example, AMD-3100, ALX40-4C, T22, T140, Met-SDF-1beta, T134, or AMD-3465. In another embodiment, the methods further comprise administering a receptor tyrosine kinase inhibitor, for example, a c-Kit inhibitor such as imatinib mesylate (Gleevec™), to the subject. In a preferred embodiment, PI3-K activity in the small cell lung cancer cells of the subject is down-regulated.

In another embodiment, the invention provides methods for identifying agents which can be used to treat small cell lung cancer comprising determining whether the agent inhibits CXCR4, for example, by determining whether the agent can compete with SDF-1α for binding to CXCR4, whether the agent can inhibit SDF-1α mediated cellular proliferation, adhesion, motility, or cell shape, and/or whether the agent inhibits PI3-K activity (e.g., phosphorylation of Akt and p70 S6 kinase).

In another embodiment, the invention provides a method for determining whether a CXCR4 inhibitor can or cannot be used to treat small cell lung cancer comprising obtaining a sample of lung cancer cells (e.g., from a cell line or a subject), and determining whether the cells express CXCR4 (e.g., by measuring CXCR4 mRNA and/or protein levels), thereby determining that the CXCR4 inhibitor can be used to treat small cell lung cancer when CXCR4 is expressed. In one embodiment, the CXCR4 inhibitor may bind to CXCR4 or SDF-1α (e.g., an antibody, an antibody fragment, or a small molecule such as AMD-3100, ALX40-4C, T22, T140, Met-SDF-1beta, T134, or AMD-3465).

In another embodiment, the invention provides a method for determining whether a combination of a CXCR4 inhibitor and a receptor tyrosine kinase inhibitor can or cannot be used to treat small cell lung cancer comprising obtaining a sample of lung cancer cells (e.g., from a cell line or a subject), and determining whether the cells express CXCR4 (e.g., by measuring CXCR4 mRNA and/or protein levels) and further determining whether the cells also express the receptor tyrosine kinase that is inhibited by said receptor tyrosine kinase inhibitor (e.g., by measuring mRNA and/or protein levels of the receptor tyrosine kinase), thereby determining that the combination of a CXCR4 inhibitor and a receptor tyrosine kinase inhibitor can be used to treat small cell lung cancer when CXCR4 and the receptor tyrosine kinase are expressed. In a preferred embodiment of this method, the receptor tyrosine kinase inhibitor is a c-Kit inhibitor such as for example imatinib mesylate (Gleevec™). The CXCR4 inhibitor may bind to CXCR4 or SDF-1α (e.g., an antibody, an antibody fragment, or a small molecule such as AMD-3100, ALX40-AC, T22, T140, Met-SDF-1beta, T134, or AMD-3465).

In another embodiment, the invention provides a method for determining whether a CXCR4 inhibitor can or cannot be used to inhibit proliferation or metastasis of small cell lung cancer comprising obtaining a sample of lung cells (e.g., from a cell line or a subject), and determining whether the cells express CXCR4 (e.g., by measuring CXCR4 mRNA and/or protein levels), thereby determining that the CXCR4 inhibitor can be used to inhibit proliferation or metastasis of small cell lung cancer when CXCR4 is expressed. In one embodiment, the CXCR4 inhibitor may bind to CXCR4 or SDF-1α (e.g., an antibody, an antibody fragment, or a small molecule such as AMD-3100, ALX40-4C, T22, T140, Met-SDF-1beta, T134, or AMD-3465).

In another embodiment, the invention provides a method for determining whether a combination of a CXCR4 inhibitor and a receptor tyrosine kinase inhibitor can or cannot be used to inhibit proliferation or metastasis of small cell lung cancer comprising obtaining a sample of lung cells (e.g., from a cell line or a subject), and determining whether the cells express CXCR4 (e.g., by measuring CXCR4 mRNA and/or protein levels) and further determining whether the cells also express the receptor tyrosine kinase that is inhibited by said receptor tyrosine kinase inhibitor (e.g., by measuring mRNA and/or protein levels of the receptor tyrosine kinase), thereby determining that the combination of a CXCR4 inhibitor and a receptor tyrosine kinase inhibitor can be used to inhibit proliferation or metastasis of small cell lung cancer when CXCR4 and the receptor tyrosine kinase are expressed. In a preferred embodiment of this method, the receptor tyrosine kinase inhibitor is a c-Kit inhibitor such as for example imatinib mesylate (Gleevec™). The CXCR4 inhibitor may bind to CXCR4 or SDF-1α (e.g., an antibody, an antibody fragment, or a small molecule such as AMD-3100, ALX40-4C, T22, T140, Met-SDF-1beta, T134, or AMD-3465).

In another embodiment, the invention provides a method for determining whether a patient would benefit from treatment with an agent that inhibits CXCR4 comprising obtaining a lung sample from the patient and determining whether CXCR4 is expressed in the sample (e.g., by measuring CXCR4 mRNA and/or protein levels).

In another embodiment, the invention provides a method for determining whether a patient would benefit from treatment with an agent that inhibits CXCR4 and an agent that inhibits c-Kit comprising obtaining a lung sample from the patient and determining whether CXCR4 and c-Kit are expressed in the sample (e.g., by measuring CXCR4 and c-Kit mRNA and/or protein levels).

In another embodiment, the invention provides a method for determining whether treatment with a CXCR4 inhibitor should be continued or discontinued in a small cell lung cancer patient, comprising obtaining two or more samples comprising tumor cells from a patient during the course of treatment, determining the level of activity in the tumor cells of CXCR4, and continuing treatment when the activity of CXCR4 does not increase during treatment. In a preferred embodiment, the level of activity in the tumor cells of CXCR4 is determined by determining the ability of SDF-1α to modulate proliferation, adhesion, motility, cell shape, or PI3-K activity in the cells.

In another embodiment, the invention provides a method for determining whether treatment with a combination of a CXCR4 inhibitor and a receptor tyrosine kinase inhibitor should be continued or discontinued in a small cell lung cancer patient, comprising obtaining two or more samples comprising tumor cells from a patient during the course of treatment, determining the level of activity in the tumor cells of CXCR4, determining the level of activity in the tumor cells of c-Kit, and continuing treatment when the activity levels of CXCR4 and/or c-Kit do not increase during treatment. In a preferred embodiment, the level of activity in the tumor cells of CXCR4 is determined by determining the ability of SDF-1α to modulate proliferation, adhesion, motility, cell shape, or PI3-K activity in the cells. In a further preferred embodiment, the level of activity in the tumor cells of c-Kit is determined by determining the ability of SCF to modulate proliferation, adhesion, motility, cell shape, or PI3-K activity in the cells.

In another embodiment, the invention provides a CXCR4 inhibitor for use in a method for the treatment of a subject. In one embodiment, the CXCR4 inhibitor may bind to CXCR4 or SDF-1α (e.g., an antibody, an antibody fragment, or a small molecule such as AMD-3100, ALX40-4C, T22, T140, Met-SDF-1beta, T134, or AMD-3465).

In another embodiment, the invention provides the use of a CXCR4 inhibitor for the preparation of a pharmaceutical composition for use in a method such as inhibiting cellular proliferation in a small cell lung cancer cell population, inhibiting cellular movement or motility in a small cell lung cancer cell population, modulating cellular adhesion in a small cell lung cancer cell population, inhibiting morphological change in a small cell lung cancer cell population, treating a subject having small cell lung cancer, or inhibiting metastasis of small cell lung cancer in a subject. In one embodiment, the CXCR4 inhibitor may bind to CXCR4 or SDF-1α (e.g., an antibody, an antibody fragment, or a small molecule such as AMD-3100, ALX40-4C, T22, T140, Met-SDF-1beta, T134, or AMD-3465).

In another embodiment, the invention provides a combination which comprises (a) a CXCR4 inhibitor and (b) a receptor tyrosine kinase inhibitor, such as especially a c-Kit inhibitor, wherein the active ingredients (a) and (b) are present in each case in free form or in the form of a pharmaceutically acceptable salt, for simultaneous, concurrent, separate or sequential use, especially in a method for the treatment of a subject, preferably in a method such as inhibiting cellular proliferation in a small cell lung cancer cell population, inhibiting cellular movement or motility in a small cell lung cancer cell population, modulating cellular adhesion in a small cell lung cancer cell population, inhibiting morphological change in a small cell lung cancer cell population, treating small cell lung cancer, or inhibiting metastasis of small cell lung cancer. In a preferred embodiment of this method, the receptor tyrosine kinase inhibitor is a c-Kit inhibitor such as for example imatinib mesylate (Gleevec™). The CXCR4 inhibitor may bind to CXCR4 or SDF-1α (e.g., an antibody, an antibody fragment, or a small molecule such as AMD-3100, ALX40-4C, T22, T140, Met-SDF-1beta, T134, or AMD-3465).

Other features and advantages of the invention will be apparent from the following detailed description and claims.

### DETAILED DESCRIPTION OF THE INVENTION

The present invention is based, at least in part, on the discovery that CXCR4 is ubiquitously expressed, and c-Kit is variably expressed, in small cell lung cancer (SCLC) cells. The present invention is further based on the discovery that treatment of SCLC cells with SDF-1α and/or SCF (the ligands for CXCR4 and c-Kit, respectively) induces proliferation and regulates adhesion, motility, and cell shape of SCLC cells. The present invention is still further based on the discovery that PI3-Kinase (PI3-K) regulates SDF-1α induced cell motility of SCLC cells, that CXCR4— and c-Kit cooperatively induce morphological changes in SCLC cells, that SDF-1α and SCF independently regulate phosphorylation of Akt and p70 S6 kinase, and that STI571 (a c-Kit inhibitor also referred to as imatinib mesylate or Gleevec™) and LY294002 (a PI3-K inhibitor) inhibit signal transduction of the CXCR4 and c-Kit pathways.

Chemokines are small cytokine-like peptides that direct various subsets of hematopoietic cells to home specific anatomical sites through interaction with their G protein-coupled receptors (Rossi, D. and Zlotnick, A. (2000) Annu. Rev. Immunol. 18:217-42; Zlotnick, A. and Yoshie, O. (2000) Immunity 12:121-7). CXCR4 is a seven-transmembrane G protein-coupled receptor and is also known as a coreceptor for human immunodeficiency virus (HIV) (Bleul, C. C. et al. (1996) Nature 382:829-3; Feng, Y. et al. (1996) Science 272:872-7; Nagasawa, T. et al. (1996) Proc. Natl. Acad. Sci. USA 93:14726-9). SDF-1α, the natural ligand for CXCR4, is a member of the CXC chemokine family which has chemotactic activity for hematopoietic progenitor cells (Aiuti, A. et al. (1997) J. Exp. Med. 185:111-20; Jo, D. Y. et al. (2000) J. Clin. Invest. 105:101-11; Nagasawa, T. et al. (1994) Proc. Natl. Acad. Sci. USA 91:2305-9; Nagasawa, T. (1996) Proc. Natl. Acad. Sci. USA 93:14726-9). Thus far, the role of interaction between chemokine receptors and cytokine receptors has not been defined for solid tumors such as SCLC.

The instant invention therefore provides methods and compositions for the identification, assessment, and therapy of SCLC. In particular, the present invention provides methods and compositions for modulating SCLC growth, proliferation, movement, motility, adhesion, morphological change, and/or metastasis by modulating CXCR4 activity in SCLC cells. In some embodiments, the activity of c-Kit may also be modulated. Accordingly, one aspect of the invention pertains to the use of CXCR4 and/or c-Kit molecules, referred to herein as CXCR4 and/or c-Kit nucleic acid and protein molecules, which comprise a family of molecules having certain conserved structural and functional features, and which play a role in or function in type I muscle formation associated activities. The term “family” when referring to the protein and nucleic acid molecules of the invention is intended to mean two or more proteins or nucleic acid molecules having a common structural domain and having sufficient amino acid or nucleotide sequence homology as defined herein. Such family members can be naturally occurring and can be from either the same or different species. For example, a family can contain a first protein of human origin, as well as other, distinct proteins of human origin or alternatively, can contain homologues of non-human origin. Members of a family may also have common functional characteristics.

Another aspect of the invention pertains to methods for treating a subject having SCLC. These methods include the step of administering a CXCR4 modulator and/or a c-Kit modulator to the subject such that treatment occurs. In a preferred embodiment, the subject being treated with a CXCR4 modulator has SCLC cells that express CXCR4. In a further preferred embodiment, a subject being treated with a CXCR4 modulator and a c-Kit modulator has SCLC cells that express both CXCR4 and c-Kit. The terms “treating” or “treatment, as used herein, refer to reduction or alleviation of at least one adverse effect or symptom of SCLC, e.g., tumor burden, tumor size, tumor cell proliferation, migration, motility, adhesion, and/or morphological change.

As used herein, a CXCR4 modulator is a molecule which can modulate CXCR4 nucleic acid expression and/or CXCR4 protein activity. For example, a CXCR4 modulator can modulate, i.e., upregulate (activate) or downregulate (suppress), CXCR4 nucleic acid expression. In another example, a CXCR4 modulator can modulate (i.e., stimulate or inhibit) CXCR4 protein activity. In a preferred embodiment, the methods of the invention use CXCR4 inhibitors, i.e., agents which inhibit CXCR4 activity. Non-limiting examples of CXCR4 inhibitors include small molecules such as AMD-3100, ALX40-4C, T22, T140, Met-SDF-1beta, T134, and AMD-3465, or small molecules or drugs identified by the screening methods described herein. CXCR4 inhibitors may also inhibit CXCR4 nucleic acid expression, for example, antisense molecules, i.e., a ribozyme, as described herein. Examples of antisense molecules which can be used to inhibit CXCR4 nucleic acid expression include antisense molecules which are complementary to a portion of the 5′ untranslated region of SEQ ID NO:1 which also includes the start codon and antisense molecules which are complementary to a portion of the 3′ untranslated region of SEQ ID NO:1. A CXCR4 modulator which inhibits CXCR4 nucleic acid expression can also be a small molecule or other drug, i.e., a small molecule or drug identified using the screening assays described herein, which inhibits CXCR4 nucleic acid expression. A CXCR4 molecule of the invention can thus also be used as a target to screen molecules, i.e., which can modulate CXCR4 activity. CXCR4 modulators may also be antibodies or antibody fragments. CXCR4 modulators may also be forms of SDF-1α which block or inhibit CXCR4 activity, e.g., dominant negative forms of SDF-1α.

As used herein, a c-Kit modulator is a molecule which can modulate c-Kit nucleic acid expression and/or c-Kit protein activity. For example, a c-Kit modulator can modulate, i.e., upregulate (activate) or downregulate (suppress), c-Kit nucleic acid expression. In another example, a c-Kit modulator can modulate (i.e., stimulate or inhibit) c-Kit protein activity. In a preferred embodiment, the methods of the invention use c-Kit inhibitors, i.e., agents which inhibit c-Kit activity. Non-limiting examples of CXCR4 inhibitors include small molecules or drugs identified by the screening methods described herein. In a preferred embodiment, a c-Kit inhibitor is a receptor tyrosine kinase inhibitor such as imatinib mesylate, also referred to interchangeably herein as STI571 or Gleevec™ (available from Novartis (Basel, Switzerland)) (Savage, D. G. and Antman, K. H. (2002) N. Engl. J. Med. 346(9):683-93; Mauro, M. J. et al. (2002) J. Clin. Oncol. 20(1):325-34; Schiffer, C. A. (2001) Semin. Oncol. 28(5 Suppl 17):34-9; Demetri, G. D. (2001) Semin. Oncol. 28(5 Suppl 17):19-26; Griffin, J. (2001) Semin. Oncol. 28(5 Suppl 17):3-8; Verweij, J. et al. (2001) Eur. J. Cancer. 37(15):1816-9; Shah, N. P. and Sawyers, C. L. (2001) Curr. Opin. Investig. Drugs. 2(3):422-3).

c-Kit inhibitors may also inhibit c-Kit nucleic acid expression, for example, antisense molecules, i.e., a ribozyme, as described herein. Examples of antisense molecules which can be used to inhibit c-Kit nucleic acid expression include antisense molecules which are complementary to a portion of the 5′ untranslated region of SEQ ID NO:3 which also includes the start codon and antsense molecules which are complementary to a portion of the 3′ untranslated region of SEQ ID NO:3. A c-Kit modulator which inhibits c-Kit nucleic acid expression can also be a small molecule or other drug, i.e., a small molecule or drug identified using the screening assays described herein, which inhibits c-Kit nucleic acid expression. A c-Kit molecule of the invention can thus also be used as a target to screen molecules, i.e., which can modulate c-Kit activity. c-Kit modulators may also be antibodies or antibody fragments. c-Kit modulators may also be forms of SCF which block or inhibit c-Kit activity, e.g., dominant negative forms of SCF.

Other aspects of the invention pertain to methods for modulating a cell associated activity. These methods include contacting the cell with an agent (or a composition which includes an effective amount of an agent) which modulates CXCR4 and/or c-Kit protein activity or CXCR4 and/or c-Kit nucleic acid expression such that a cell associated activity is altered relative to a cell associated activity of the cell in the absence of the agent. As used herein, “a cell associated activity” refers to a normal or abnormal activity or function of a cell, e.g., an SCLC cell. Examples of cell associated activities include proliferation, growth, movement, motility, adhesion, and/or morphological change. In a preferred embodiment, the cell associated activity is metastasis. The term “altered” as used herein refers to a change, i.e., an increase or decrease, of a cell associated activity. In a preferred embodiment, the agent inhibits CXCR4 and/or c-Kit protein activity or CXCR4 and/or c-Kit nucleic acid expression. Examples of such inhibitory agents include a nucleic acid molecule encoding a dominant negative CXCR4 and/or c-Kit protein, a dominant negative SDF-1α and/or SCF protein, an antisense CXCR4 and/or c-Kit nucleic acid molecule, an anti-CXCR4 and/or anti-c-Kit antibody or antibody fragment, and a modulatory agent which inhibits CXCR4 and/or c-Kit protein activity or and/or c-Kit CXCR4 nucleic acid expression and which may be identified using the drug screening assays described herein. These modulatory methods can be performed in vitro (i.e., by culturing the cell with the agent) or, alternatively, in vivo (i.e., by administering the agent to a subject). In a preferred embodiment, the modulatory methods are performed in vivo, i.e., the cell is present within a subject, i.e., a mammal, i.e., a human, and the subject has SCLC.

The methods of the present invention may therefore: 1) modulate growth of SCLC cells; 2) modulate proliferation of SCLC cells; 3) modulate movement of SCLC cells; 4) modulate motility of SCLC cells; 5) modulate adhesion of SCLC cells; 6) modulate cellular shape and/or morphological change of SCLC cells; 7) modulate metastasis of SCLC cells; 8) modulate CXCR4 activity; 9) modulate CXCR4 and c-Kit activity; 10) modulate CXCR4 binding to SDF-1α; 11) modulate c-Kit binding to SCF; and/or 12) modulate PI3-K activity.

A nucleic acid molecule, a protein, a CXCR4 and/or c-Kit modulator, a compound etc. used in the methods of treatment can be incorporated into an appropriate pharmaceutical composition described herein and administered to the subject through a route which allows the molecule, protein, modulator, or compound etc. to perform its intended function. Examples of routes of administration are also described herein.

The nucleotide sequence of the human CXCR4 cDNA and the predicted amino acid sequence of the human CXCR4 protein are shown in SEQ ID NOs:1 and 2, respectively. The human CXCR4 gene, which is approximately 1679 nucleotides in length, encodes a full length protein having a molecular weight of approximately 38.7 kD and which is approximately 352 amino acid residues in length. Further description of the human CXCR4 nucleic acid and polypeptide sequences can be found in GenBank Accession Nos. NM_{—}003467 and NP_{—}003458, respectively; as well as in Federsppiel, B. et al. (1993) Genomics 16(3):707-712; Herzog, H. et al. (1993) DNA Cell Biol. 12(6):465-471; Jazin, E. E. et al. (1993) Regul. Pept 47(3):247-258; Nomura, H. et al. (1993) Int. Immunol. 5(10):1239-1249; Loetscher, M. et al. (1994) J. Biol. Chem. 269(1):232-237; Moriuchi, M. et al. (1997) J. Immunol. 159(9):4322-4329; Caruz, A. et al. (1998) FEBS Lett. 426(2):271-278; and Wegner, S. A. et al. (1998) J. Biol. Chem. 273(8):4754-4760.

The nucleotide sequence of the human c-Kit cDNA and the predicted amino acid sequence of the human c-Kit protein are shown in SEQ ID NOs:3 and 4, respectively. The human c-Kit gene, which is approximately 5084 nucleotides in length, encodes a full length protein having a molecular weight of approximately 107.4 kD and which is approximately 976 amino acid residues in length. Further description of the human c-Kit nucleic acid and polypeptide sequences can be found in GenBank Accession Nos. NM_{—}000222 and NP_{—}000213, respectively; as well as in Yarden, Y. et al. (1987) EMBO J. 6(11):3341-3351; Andre, C. et al. (1992) Oncogene 7(4):685-691; Giebel, L. B. et al. (1992) Oncogene 7(11):2207-2217; Yamamoto, K. et al. (1993) Jpn. J. Cancer Res. 84(11):1136-1144; Toyota, M. et al. (1994) Cancer Res. 54(1):272-275; Zhu W. M. et al. (1994) Leuk. Lymphoma 12(5-6):441-447; and Andre, C. (1997) Genomics 39(2):216-226.

Various aspects of the invention are described in further detail in the following subsections:

I. Isolated Nucleic Acid Molecules

One aspect of the invention pertains to methods utilizing isolated nucleic acid molecules that encode CXCR4 and/or c-Kit, or biologically active portions thereof, as well as nucleic acid fragments sufficient for use as hybridization probes to identify CXCR4 and/or c-Kit encoding nucleic acid (i.e., CXCR4 and/or c-Kit mRNA). As used herein, the term “nucleic acid molecule” is intended to include DNA molecules (i.e., cDNA or genomic DNA) and RNA molecules (i.e., mRNA) and analogs of the DNA or RNA generated using nucleotide analogs. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated CXCR4 and/or c-Kit nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (i.e., a lung cell). Moreover, an “isolated” nucleic acid molecule, such as a cDNA molecule, can be substantially free of other cellular material, or culture medium when produced by recombinant techniques, or chemical precursors or other chemicals when chemically synthesized.

A nucleic acid molecule of the present invention, i.e., a nucleic acid molecule having the nucleotide sequence of SEQ ID NO:1 or 3 or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1 or 3 or a portion thereof (e.g., 50, 100, 200, 300, 400, 450, 500, or more nucleotides), can be isolated using standard molecular biology techniques and the sequence information provided herein. For example, a human CXCR4 and/or c-Kit cDNA can be isolated from a human SCLC cell line (from Stratagene, LaJolla, Calif., or Clontech, Palo Alto, Calif.) using all or portion of SEQ ID NO:1 or 3 as a hybridization probe and standard hybridization techniques (i.e., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of SEQ ID NO:1 or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1 or 3 can be isolated by the polymerase chain reaction using oligonucleotide primers designed based upon the sequence of SEQ ID NO:1 or 3 or the homologous nucleotide sequence. For example, mRNA can be isolated from lung cells or lung tumor cells (i.e., by the guanidinium-thiocyanate extraction procedure of Chirgwin et al. (1979) Biochemistry 18:5294-5299) and cDNA can be prepared using reverse transcriptase (i.e., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for PCR amplification can be designed based upon the nucleotide sequence shown in SEQ ID NO:1 or 3 or to the homologous nucleotide sequence. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to a CXCR4 and/or c-Kit nucleotide sequence can be prepared by standard synthetic techniques, i.e., using an automated DNA synthesizer.

In a preferred embodiment, an isolated nucleic acid molecule of the invention comprises the nucleotide sequence shown in SEQ ID NO:1 or 3 or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1 or 3. The sequence of SEQ ID NO:1 corresponds to the human CXCR4 cDNA. This cDNA comprises sequences encoding the CXCR4 protein (i.e., “the coding region”, from nucleotides 89 to 1144), as well as 5′ untranslated sequences (nucleotides 1 to 88) and 3′ untranslated sequences (nucleotides 1145 to 1679). Alternatively, the nucleic acid molecule can comprise only the coding region of SEQ ID NO:1 (i.e., nucleotides 89 to 1144). The sequence of SEQ ID NO:3 corresponds to the human c-Kit cDNA. This cDNA comprises sequences encoding the c-Kit protein (i.e., “the coding region”, from nucleotides 22 to 2949), as well as 5′ untranslated sequences (nucleotides 1 to 21) and 3′ untranslated sequences (nucleotides 2950 to 5084). Alternatively, the nucleic acid molecule can comprise only the coding region of SEQ ID NO:3 (i.e., nucleotides 22 to 2949).

In another preferred embodiment, an isolated nucleic acid molecule of the invention comprises a nucleic acid molecule which is a complement of the nucleotide sequence shown in SEQ ID NO:1 or 3 or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1 or 3. A nucleic acid molecule which is complementary to the nucleotide sequence shown in SEQ ID NO:1 or 3 or to a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1 or 3 is one which is sufficiently complementary to the nucleotide sequence shown in SEQ ID NO:1 or 3 or to the homologous sequence such that it can hybridize to the nucleotide sequence shown in SEQ ID NO:1 or 3 or to the homologous sequence, thereby forming a stable duplex.

In still another preferred embodiment, an isolated nucleic acid molecule of the invention comprises a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1 or 3 or a portion of this nucleotide sequence. In an additional preferred embodiment, an isolated nucleic acid molecule of the invention comprises a nucleotide sequence which hybridizes, i.e., hybridizes under stringent conditions, to the nucleotide sequence shown in SEQ ID NO:1 or 3 or to a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1 or 3.

Moreover, the nucleic acid molecule of the invention can comprise only a portion of the coding region of SEQ ID NO:1 or 3 or the coding region of a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1 or 3, for example a fragment which can be used as a probe or primer or a fragment encoding a biologically active portion of CXCR4 and/or c-Kit. The nucleotide sequence determined from the cloning of the CXCR4 and/or c-Kit gene from a mouse or human allows for the generation of probes and primers designed for use in identifying and/or cloning other CXCR4 and/or c-Kit family members, as well as CXCR4 and/or c-Kit homologues in other cell types, i.e. from other tissues, as well as CXCR4 and/or c-Kit homologues from other mammals such as rats or monkeys. The probe/primer typically comprises substantially purified oligonucleotide. The oligonucleotide typically comprises a region of nucleotide sequence that hybridizes under stringent conditions to at least about 12, preferably at least about 25, more preferably about 40, 50 or 75 consecutive nucleotides of SEQ ID NO:1 or SEQ ID NO:3 sense, an anti-sense sequence of SEQ ID NO:1 or SEQ ID NO:3, or naturally occurring mutants thereof. Primers based on the nucleotide sequence in SEQ ID NO:1 or SEQ ID NO:3 can be used in PCR reactions to clone CXCR4 and/or c-Kit homologues.

Probes based on the CXCR4 and/or c-Kit nucleotide sequences can be used to detect transcripts or genomic sequences encoding the same or homologous proteins. In preferred embodiments, the probe further comprises a label group attached thereto, i.e. the label group can be a radioisotope, a fluorescent compound, an enzyme, or an enzyme co-factor. Such probes can be used as a part of a diagnostic test kit for identifying cells or tissue (e.g., lung cells or lung tumor cells) which express or misexpress a CXCR4 and/or c-Kit protein, such as by measuring a level of a CXCR4 and/or c-Kit encoding nucleic acid in a sample of cells (e.g., lung cells) from a subject, i.e., detecting CXCR4 and/or c-Kit mRNA levels or determining whether a genomic CXCR4 and/or c-Kit gene has been mutated or deleted.

In one embodiment, the nucleic acid molecule of the invention encodes a protein or portion thereof which includes an amino acid sequence which is sufficiently homologous to an amino acid sequence of SEQ ID NO:2 or 4 such that the protein or portion thereof maintains one or more of the following biological activities: 1) it can modulate growth of SCLC cells; 2) it can modulate proliferation of SCLC cells; 3) it can modulate movement of SCLC cells; 4) it can modulate motility of SCLC cells; 5) it can modulate adhesion of SCLC cells; 6) it can modulate cellular shape and/or morphological change of SCLC cells; 7) it can modulate metastasis of SCLC cells; 8) it can modulate CXCR4 activity; 9) it can modulate CXCR4 and c-Kit activity; 10) it can modulate CXCR4 binding to SDF-1α; 11) it can modulate c-Kit binding to SCF; and/or 12) it can modulate PI3-K activity.

As used herein, the language “sufficiently homologous” refers to proteins or portions thereof which have amino acid sequences which include a minimum number of identical or equivalent (i.e., an amino acid residue which has a similar side chain as an amino acid residue in SEQ ID NO:2 or 4) amino acid residues to an amino acid sequence of SEQ ID NO:2 or 4 such that the protein or portion thereof maintains one or more of the following biological activities: 1) it can modulate growth of SCLC cells; 2) it can modulate proliferation of SCLC cells; 3) it can modulate movement of SCLC cells; 4) it can modulate motility of SCLC cells; 5) it can modulate adhesion of SCLC cells; 6) it can modulate cellular shape and/or morphological change of SCLC cells; 7) it can modulate metastasis of SCLC cells; 8) it can modulate CXCR4 activity; 9) it can modulate CXCR4 and c-Kit activity; 10) it can modulate CXCR4 binding to SDF-1α; 11) it can modulate c-Kit binding to SCF; and/or 12) it can modulate PI3-K activity.

In another embodiment, the protein is at least about 50%, preferably at least about 60%, more preferably at least about 70%, yet more preferably at least about 80%, still more preferably at least about 90%, and most preferably at least about 95% or more homologous to the entire amino acid sequence of SEQ ID NO:2 or 4.

Portions of proteins encoded by the CXCR4 and/or c-Kit nucleic acid molecule of the invention are preferably biologically active portions of the CXCR4 and/or c-Kit protein. As used herein, the term “biologically active portion of CXCR4 and/or c-Kit” is intended to include a portion, i.e., a domain/motif, of CXCR4 and/or c-Kit that has one or more of the following activities: 1) it can modulate growth of SCLC cells; 2) it can modulate proliferation of SCLC cells; 3) it can modulate movement of SCLC cells; 4) it can modulate motility of SCLC cells; 5) it can modulate adhesion of SCLC cells; 6) it can modulate cellular shape and/or morphological change of SCLC cells; 7) it can modulate metastasis of SCLC cells; 8) it can modulate CXCR4 activity; 9) it can modulate CXCR4 and c-Kit activity; 10) it can modulate CXCR4 binding to SDF-1α; 11) it can modulate c-Kit binding to SCF; and/or 12) it can modulate PI3-K activity.

Standard binding assays, i.e., immunoprecipitations and yeast two-hybrid assays, as described herein, can be performed to determine the ability of a CXCR4 or c-Kit protein or a biologically active portion thereof to interact with (i.e., bind to) SDF-1α or SCF, respectively.

In one embodiment, the biologically active portion of CXCR4 and/or c-Kit comprises at least one domain or motif. In one embodiment, the biologically active portion of the protein which includes the domain or motif can modulate proliferation or metastasis of SCLC cells or can modulate PI3-K signaling. These domains are described herein. Additional nucleic acid fragments encoding biologically active portions of CXCR4 and/or c-Kit can be prepared by isolating a portion of SEQ ID NO:1 or 3 or a homologous nucleotide sequence, expressing the encoded portion of CXCR4 and/or c-Kit protein or peptide (i.e., by recombinant expression in vitro) and assessing the activity of the encoded portion of CXCR4 and/or c-Kit protein or peptide.

The invention further encompasses nucleic acid molecules that differ from the nucleotide sequence shown in SEQ ID NO:1 or 3 (and portions thereof) due to degeneracy of the genetic code and thus encode the same CXCR4 and/or c-Kit protein as that encoded by the nucleotide sequence shown in SEQ ID NO:1 or 3. In another embodiment, an isolated nucleic acid molecule of the invention has a nucleotide sequence encoding a protein having an amino acid sequence shown in SEQ ID NO:2 or 4 or a protein having an amino acid sequence which is at least about 50%, preferably at least about 60%, more preferably at least about 70%, yet more preferably at least about 80%, still more preferably at least about 90%, and most preferably at least about 95% or more homologous to the amino acid sequence of SEQ ID NO:2 or 4.

In addition to the human CXCR4 and c-Kit nucleotide sequences shown in SEQ ID NO:1 and 3, respectively, it will be appreciated by those skilled in the art that DNA sequence polymorphisms that lead to changes in the amino acid sequences of CXCR4 and/or c-Kit may exist within a population (i.e., a mammalian population, i.e., a human population). Such genetic polymorphism in the CXCR4 or c-Kit gene may exist among individuals within a population due to natural allelic variation. As used herein, the terms “gene” and “recombinant gene” refer to nucleic acid molecules comprising an open reading frame encoding a CXCR4 or c-Kit protein, preferably a mammalian, i.e., human, CXCR4 or c-Kit protein. Such natural allelic variations can typically result in 1-5% variance in the nucleotide sequence of the CXCR4 or c-Kit gene. Any and all such nucleotide variations and resulting amino acid polymorphisms in CXCR4 and/or c-Kit that are the result of natural allelic variation and that do not alter the functional activity of CXCR4 and/or c-Kit are intended to be within the scope of the methods of the invention. Moreover, nucleic acid molecules encoding CXCR4 and/or c-Kit proteins from other species, and thus which have a nucleotide sequence which differs from the human or mouse sequences of SEQ ID NO:1 or 3 are intended to be within the scope of the invention. Nucleic acid molecules corresponding to natural allelic variants and homologues of the mouse or human CXCR4 and/or c-Kit cDNAs of the invention can be isolated based on their homology to the human CXCR4 and/or c-Kit nucleic acid sequences disclosed herein using the human cDNA, or a portion thereof, as a hybridization probe according to standard hybridization techniques under stringent hybridization conditions (as described herein).

Moreover, nucleic acid molecules encoding other CXCR4 and/or c-Kit family members and thus which have a nucleotide sequence which differs from the CXCR4 and/or c-Kit sequences of SEQ ID NO:1 or 3 are intended to be within the scope of the invention. For example, the use of alternately-spliced isoforms of CXCR4 and/or c-Kit, other CXCR4 and/or c-Kit family members, or CXCR4 and/or c-Kit members from other species, and thus which have a nucleotide sequence which differs from the CXCR4 and c-Kit sequences of SEQ ID NO:1 and 3 are intended to be within the scope of the invention. For example, rat or monkey CXCR4 or c-Kit cDNA can be identified based on the nucleotide sequence of a human CXCR4 or c-Kit, respectively.

Accordingly, in another embodiment, an isolated nucleic acid molecule of the invention is at least 15 nucleotides in length and hybridizes under stringent conditions to the nucleic acid molecule comprising the nucleotide sequence of SEQ ID NO:1 or 3 or a nucleotide sequence which is about 60%, preferably at least about 70%, more preferably at least about 80%, still more preferably at least about 90%, and most preferably at least about 95% or more homologous to the nucleotide sequence of SEQ ID NO:1 or 3. In other embodiments, the nucleic acid is at least 30, 50, 100, 250 or 500 nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of SEQ ID NO:1 or SEQ ID NO:3 corresponds to a naturally-occurring nucleic acid molecule. As used herein, a “naturally-occurring” nucleic acid molecule refers to an RNA or DNA molecule having a nucleotide sequence that occurs in nature (i.e., encodes a natural protein). In one embodiment, the nucleic acid encodes a natural human CXCR4 or c-Kit.

In addition to naturally-occurring allelic variants of the CXCR4 or c-Kit sequence that may exist in the population, the skilled artisan will further appreciate that changes can be introduced by mutation into the nucleotide sequence of SEQ ID NO:1 or 3, thereby leading to changes in the amino acid sequence of the encoded CXCR4 or c-Kit protein, without altering the functional ability of the CXCR4 or c-Kit protein. For example, nucleotide substitutions leading to amino acid substitutions at “non-essential” amino acid residues can be made in the sequence of SEQ ID NO:1 or 3. A non-essential” amino acid residue is a residue that can be altered from the wild-type sequence of CXCR4 or c-Kit (i.e., the sequence of SEQ ID NO:2 or 4, respectively) without altering the activity of CXCR4 or c-Kit, whereas an “essential” amino acid residue is required for CXCR4 or c-Kit activity. For example, amino acid residues involved in the interaction of CXCR4 or c-Kit to binding partners or target molecules (e.g., SDF-1α or SCF, respectively) are most likely essential residues of CXCR4 or c-Kit. Other amino acid residues, however, (i.e., those that are not conserved or only semi-conserved between mouse and human) may not be essential for activity and thus are likely to be amenable to alteration without altering CXCR4 or c-Kit activity. Furthermore, amino acid residues that are essential for CXCR4 or c-Kit functions related to SCLC proliferation or metastasis, but not essential for CXCR4 or c-Kit functions related to HIV infection, are likely to be amenable to alteration.

Accordingly, another aspect of the invention pertains to nucleic acid molecules encoding CXCR4 or c-Kit proteins that contain changes in amino acid residues that are not essential for CXCR4 or c-Kit activity. Such CXCR4 and c-Kit proteins differ in amino acid sequence from SEQ ID NOs:2 and 4 yet retain at least one of the CXCR4 or c-Kit activities described herein. In one embodiment, the isolated nucleic acid molecule comprises a nucleotide sequence encoding a protein, wherein the protein comprises an amino acid sequence at least about 60% homologous to the amino acid sequence of SEQ ID NO:2 or 4 and is capable of modulating proliferation or metastasis. Preferably, the protein encoded by the nucleic acid molecule is at least about 70% homologous, preferably at least about 80-85% homologous, still more preferably at least about 90%, and most preferably at least about 95% homologous to the amino acid sequence of SEQ ID NO:2 or 4.

“Sequence identity or homology”, as used herein, refers to the sequence similarity between two polypeptide molecules or between two nucleic acid molecules. When a position in both of the two compared sequences is occupied by the same base or amino acid monomer subunit, i.e., if a position in each of two DNA molecules is occupied by adenine, then the molecules are homologous or sequence identical at that position. The percent of homology or sequence identity between two sequences is a function of the number of matching or homologous identical positions shared by the two sequences divided by the number of positions compared ×100. For example, if 6 of 10, of the positions in two sequences are the same then the two sequences are 60% homologous or have 60% sequence identity. By way of example, the DNA sequences ATTGCC and TATGGC share 50% homology or sequence identity. Generally, a comparison is made when two sequences are aligned to give maximum homology. Unless otherwise specified “loop out regions”, i.e., those arising from, from deletions or insertions in one of the sequences are counted as mismatches.

The comparison of sequences and determination of percent homology between two sequences can be accomplished using a mathematical algorithm. Preferably, the alignment can be performed using the Clustal Method. Multiple alignment parameters include GAP Penalty=10, Gap Length Penalty=10. For DNA alignments, the pairwise alignment parameters can be Htuple=2, Gap penalty=5, Window=4, and Diagonal saved=4. For protein alignments, the pairwise alignment parameters can be Ktuple=1, Gap penalty=3, Window=5, and Diagonals Saved=5.

In a preferred embodiment, the percent identity between two amino acid sequences is determined using the Needleman and Wunsch (J. Mol. Biol. (48):444-453 (1970)) algorithm which has been incorporated into the GAP program in the GCG software package (available online), using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available online), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0) (available online), using a PAM120 weight residue table, a gap length penalty of 12 and a gap penalty of 4.

An isolated nucleic acid molecule encoding a CXCR4 protein homologous to the protein of SEQ ID NO:2 or 4 can be created by introducing one or more nucleotide substitutions, additions or deletions into the nucleotide sequence of SEQ ID NO:1 or 3 or a homologous nucleotide sequence such that one or more amino acid substitutions, additions or deletions are introduced into the encoded protein. Mutations can be introduced into SEQ ID NO:1 or 3 or the homologous nucleotide sequence by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (i.e., lysine, arginine, histidine), acidic side chains (i.e., aspartic acid, glutamic acid), uncharged polar side chains (i.e., glycine, asparagine, glutamine, serine, threonine, tyrosine, cysteine), nonpolar side chains (i.e., alanine, valine, leucine, isoleucine, proline, phenylalanine, methionine, tryptophan), beta-branched side chains (i.e., threonine, valine, isoleucine) and aromatic side chains (i.e., tyrosine, phenylalanine, tryptophan, histidine). Thus, a predicted nonessential amino acid residue in CXCR4 or c-Kit is preferably replaced with another amino acid residue from the same side chain family. Alternatively, in another embodiment, mutations can be introduced randomly along all or part of a CXCR4 or c-Kit coding sequence, such as by saturation mutagenesis, and the resultant mutants can be screened for a CXCR4 or c-Kit activity described herein to identify mutants that retain CXCR4 or c-Kit activity. Following mutagenesis of SEQ ID NO:1 or 3, the encoded protein can be expressed recombinantly (as described herein) and the activity of the protein can be determined using, for example, assays described herein.

In addition to the nucleic acid molecules encoding CXCR4 and c-Kit proteins described above, another aspect of the invention pertains to isolated nucleic acid molecules which are antisense thereto. An “antisense” nucleic acid comprises a nucleotide sequence which is complementary to a “sense” nucleic acid encoding a protein, i.e., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire CXCR4 or c-Kit coding strand, or to only a portion thereof. In one embodiment, an antisense nucleic acid molecule is antsense to a “coding region” of the coding strand of a nucleotide sequence encoding CXCR4 or c-Kit. The term “coding region” refers to the region of the nucleotide sequence comprising codons which are translated into amino acid residues (i.e., the entire coding region of SEQ ID NO:1 comprises nucleotides 89-1144, and the entire coding region of SEQ ID NO:4 comprises nucleotides 22 to 2949). In another embodiment, the antisense nucleic acid molecule is antisense to a “noncoding region” of the coding strand of a nucleotide sequence encoding CXCR4 or c-Kit. The term “noncoding region” refers to 5′ and 3′ sequences which flank the coding region that are not translated into amino acids (i.e., also referred to as 5′ and 3′ untranslated regions).

Given the coding strand sequences encoding CXCR4 and c-Kit disclosed herein, antisense nucleic acids of the invention can be designed according to the rules of Watson and Crick base pairing. The antisense nucleic acid molecule can be complementary to the entire coding region of CXCR4 or c-Kit mRNA, but more preferably is an oligonucleotide which is antisense to only a portion of the coding or noncoding region of CXCR4 or c-Kit mRNA. For example, the antisense oligonucleotide can be complementary to the region surrounding the translation start site of CXCR4 or c-Kit mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (i.e., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, i.e., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6 isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6 isopentenyladenine, uracil-5-oxyacetic acid (v), wybutoxosine, pseudouracil, queosine, 2-thiocytosine, 5methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antsense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a CXCR4 or c-Kit protein to thereby inhibit expression of the protein, i.e., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, i.e., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (i.e., hammerhead ribozymes (described in Haseloff and Gerlach (1988) Nature 334:585-591)) can be used to catalytically cleave CXCR4 or c-Kit mRNA transcripts to thereby inhibit translation of CXCR4 or c-Kit mRNA A ribozyme having specificity for a CXCR4 or c-Kit encoding nucleic acid can be designed based upon the nucleotide sequence of a CXCR4 or c-Kit cDNA disclosed herein (i.e., SEQ ID NO:1 or 3). For example, a derivative of a Tetrahymena L-19 IVS RNA can be constructed in which the nucleotide sequence of the active site is complementary to the nucleotide sequence to be cleaved in a CXCR4 or c-Kit encoding mRNA. See, i.e., Cech et al. U.S. Pat. No. 4,987,071 and Cech et al. U.S. Pat. No. 5,116,742. Alternatively, CXCR4 mRNA can be used to select a catalytic RNA having a specific ribonuclease activity from a pool of RNA molecules. See, i.e., Bartel, D. and Szostak, J. W. (1993) Science 261:1411-1418.

Alternatively, CXCR4 or c-Kit gene expression can be inhibited by targeting nucleotide sequences complementary to the regulatory region of the CXCR4 or c-Kit (i.e., the CXCR4 or c-Kit promoter and/or enhancers) to form triple helical structures that prevent transcription of the CXCR4 or c-Kit gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

II. Isolated CXCR4 and c-Kit Proteins and Anti-CXCR4 and c-Kit Antibodies

Another aspect of the invention pertains to the use of isolated CXCR4 or c-Kit proteins, and biologically active portions thereof, as well as peptide fragments suitable for use as immunogens to raise anti-CXCR4 and c-Kit antibodies. An “isolated” or “purified” protein or biologically active portion thereof is substantially free of cellular material when produced by recombinant DNA techniques, or chemical precursors or other chemicals when chemically synthesized. The language “substantially free of cellular material” includes preparations of CXCR4 or c-Kit protein in which the protein is separated from cellular components of the cells in which it is naturally or recombinantly produced. In one embodiment, the language “substantially free of cellular material includes preparations of CXCR4 or c-Kit protein having less than about 30% (by dry weight) of non-CXCR4 or c-Kit protein (also referred to herein as a “contaminating protein”), more preferably less than about 20% of non-CXCR4 or c-Kit protein, still more preferably less than about 10% of non-CXCR4 or c-Kit protein, and most preferably less than about 5% non-CXCR4 or c-Kit protein. When the CXCR4 or c-Kit protein or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations of CXCR4 or c-Kit protein in which the protein is separated from chemical precursors or other chemicals which are involved in the synthesis of the protein. In one embodiment, the language “substantially free of chemical precursors or other chemicals” includes preparations of CXCR4 or c-Kit protein having less than about 30% (by dry weight) of chemical precursors or non-CXCR4 or c-Kit chemicals, more preferably less than about 20% chemical precursors or non-CXCR4 or c-Kit chemicals, still more preferably less than about 10% chemical precursors or non-CXCR4 or c-Kit chemicals, and most preferably less than about 5% chemical precursors or non-CXCR4 or c-Kit chemicals. In preferred embodiments, isolated proteins or biologically active portions thereof lack contaminating proteins from the same animal from which the CXCR4 or c-Kit protein is derived. Typically, such proteins are produced by recombinant expression of, for example, a human CXCR4 or c-Kit protein in a nonhuman cell.

An isolated CXCR4 protein or a portion thereof of the invention has one or more of the following biological activities: 1) it can modulate growth of SCLC cells; 2) it can modulate proliferation of SCLC cells; 3) it can modulate movement of SCLC cells; 4) it can modulate motility of SCLC cells; 5) it can modulate adhesion of SCLC cells; 6) it can modulate cellular shape and/or morphological change of SCLC cells; 7) it can modulate metastasis of SCLC cells; 8) it can modulate CXCR4 activity; 9) it can modulate CXCR4 and c-Kit activity; 10) it can modulate CXCR4 binding to SDF-1α; 11) it can modulate c-Kit binding to SCF; and/or 12) it can modulate PI3-K activity.

In preferred embodiments, the protein or portion thereof comprises an amino acid sequence which is sufficiently homologous to an amino acid sequence of SEQ ID NO:2 or 4 such that the protein or portion thereof maintains the ability to modulate any of the above-described CXCR4 or c-Kit activities. The portion of the protein is preferably a biologically active portion as described herein. In another preferred embodiment, the CXCR4 or c-Kit protein has an amino acid sequence shown in SEQ ID NO:2 or 4, or an amino acid sequence which is at least about 50%, preferably at least about 60%, more preferably at least about 70%, yet more preferably at least about 80%, still more preferably at least about 90%, and most preferably at least about 95% or more homologous to the amino acid sequence shown in SEQ ID NO:2 or 4. In yet another preferred embodiment, the CXCR4 or c-Kit protein has an amino acid sequence which is encoded by a nucleotide sequence which hybridizes, i.e., hybridizes under stringent conditions, to the nucleotide sequence of SEQ ID NO:1 or 3 or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NO:1 or 3. The preferred CXCR4 or c-Kit proteins used in the methods of the present invention also preferably possess at least one of the CXCR4 or c-Kit biological activities described herein. For example, a preferred CXCR4 or c-Kit protein used in the methods of the present invention includes an amino acid sequence encoded by a nucleotide sequence which hybridizes, i.e., hybridizes under stringent conditions, to the nucleotide sequence of SEQ ID NO:1 or 3 and which can modulate any of the above-described CXCR4 or c-Kit activities.

In other embodiments, the CXCR4 or c-Kit protein is substantially homologous to the amino acid sequence of SEQ ID NO:2 or 4 and retains the functional activity of the protein of SEQ ID NO:2 or 4, yet differs in amino acid sequence due to natural allelic variation or mutagenesis, as described in detail in subsection I above. Accordingly, in another embodiment, the CXCR4 or c-Kit protein is a protein which comprises an amino acid sequence which is at least about 50%, preferably at least about 60%, more preferably at least about 70%, yet more preferably at least about 80%, still more preferably at least about 90%, and most preferably at least about 95% or more homologous to the amino acid sequence of SEQ ID NO:2 or 4.

Biologically active portions of the CXCR4 or c-Kit protein include peptides comprising amino acid sequences derived from the amino acid sequence of the CXCR4 or c-Kit protein, i.e., the amino acid sequence shown in SEQ ID NO:2 or 4 or the amino acid sequence of a protein homologous to the CXCR4 or c-Kit protein, which include fewer amino acids than the full length CXCR4 or c-Kit protein or the full length protein which is homologous to the CXCR4 or c-Kit protein, and exhibit at least one activity of the CXCR4 or c-Kit protein. Typically, biologically active portions (peptides, i.e., peptides which are, for example, 5, 10, 15, 20, 30, 35, 36, 37, 38, 39, 40, 50, 100 or more amino acids in length) comprise a domain or motif, with at least one activity of the CXCR4 or c-Kit protein. Moreover, other biologically active portions, in which other regions of the protein are deleted, can be prepared by recombinant techniques and evaluated for one or more of the activities described herein. Preferably, the biologically active portions of the CXCR4 or c-Kit protein include one or more selected domains/motifs or portions thereof having biological activity.

CXCR4 or c-Kit proteins are preferably produced by recombinant DNA techniques. For example, a nucleic acid molecule encoding the protein is cloned into an expression vector (as described above), the expression vector is introduced into a host cell (as described above) and the CXCR4 or c-Kit protein is expressed in the host cell. The CXCR4 or c-Kit protein can then be isolated from the cells by an appropriate purification scheme using standard protein purification techniques. Alternative to recombinant expression, a CXCR4 or c-Kit protein, polypeptide, or peptide can be synthesized chemically using standard peptide synthesis techniques. Moreover, native CXCR4 or c-Kit protein can be isolated from cells (i.e., lung cells or lung tumor cells), for example using an anti-CXCR4 or c-Kit antibody (described further below).

The invention also provides for the use of CXCR4 or c-Kit chimeric or fusion proteins. As used herein, a CXCR4 or c-Kit “chimeric protein” or “fusion protein” comprises a CXCR4 or c-Kit polypeptide operatively linked to a non-CXCR4 or c-Kit polypeptide. A “CXCR4 or c-Kit polypeptide” refers to a polypeptide having an amino acid sequence corresponding to CXCR4 or c-Kit, respectively, whereas a “non-CXCR4 or c-Kit polypeptide” refers to a polypeptide having an amino acid sequence corresponding to a protein which is not substantially homologous to the CXCR4 or c-Kit protein, i.e., a protein which is different from the CXCR4 or c-Kit protein and which is derived from the same or a different organism. Within the fusion protein, the term “operatively linked” is intended to indicate that the CXCR4 or c-Kit polypeptide and the non-CXCR4 or c-Kit polypeptide are fused in-frame to each other. The non-CXCR4 or c-Kit polypeptide can be fused to the N-terminus or C-terminus of the CXCR4 or c-Kit polypeptide. For example, in one embodiment the fusion protein is a GST-CXCR4 or c-Kit fusion protein in which the CXCR4 or c-Kit sequences are fused to the C-terminus of the GST sequences. Such fusion proteins can facilitate the purification of recombinant CXCR4 or c-Kit. In another embodiment, the fusion protein is a CXCR4 or c-Kit protein containing a heterologous signal sequence at its N-terminus. In certain host cells (i.e., mammalian host cells), expression and/or secretion of CXCR4 or c-Kit can be increased through use of a heterologous signal sequence.

Preferably, a CXCR4 or c-Kit chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (i.e., a GST polypeptide). A CXCR4 or c-Kit encoding nucleic acid can be cloned into such an expression vector such that the fusion moiety is linked in-frame to the CXCR4 or c-Kit protein.

The present invention also pertains to the use of homologues of the CXCR4 or c-Kit proteins which function as either a CXCR4 or c-Kit agonist (mimetic) or a CXCR4 or c-Kit antagonist. In a preferred embodiment, the CXCR4 or c-Kit agonists and antagonists stimulate or inhibit, respectively, a subset of the biological activities of the naturally occurring form of the CXCR4 or c-Kit protein. Thus, specific biological effects can be elicited by treatment with a homologue of limited function. In one embodiment, treatment of a subject with a homologue having a subset of the biological activities of the naturally occurring form of the protein has fewer side effects in a subject relative to treatment with the naturally occurring form of the CXCR4 or c-Kit protein.

Homologues of the CXCR4 or c-Kit protein can be generated by mutagenesis, i.e., discrete point mutation or truncation of the CXCR4 or c-Kit protein. As used herein, the term “homologue” refers to a variant form of the CXCR4 or c-Kit protein which acts as an agonist or antagonist of the activity of the CXCR4 or c-Kit protein. An agonist of the CXCR4 or c-Kit protein can retain substantially the same, or a subset, of the biological activities of the CXCR4 or c-Kit protein. An antagonist of the CXCR4 or c-Kit protein can inhibit one or more of the activities of the naturally occurring form of the CXCR4 or c-Kit protein, by, for example, competitively binding to a downstream or upstream member of the CXCR4 or c-Kit cascade which includes the CXCR4 or c-Kit protein. Thus, the mammalian CXCR4 or c-Kit protein and homologues thereof of the present invention can be, for example, either positive or negative regulators of SCLC proliferation or metastasis.

In an alternative embodiment, homologues of the CXCR4 or c-Kit protein can be identified by screening combinatorial libraries of mutants, i.e., truncation mutants, of the CXCR4 or c-Kit protein for CXCR4 or c-Kit protein agonist or antagonist activity. In one embodiment, a variegated library of CXCR4 or c-Kit variants is generated by combinatorial mutagenesis at the nucleic acid level and is encoded by a variegated gene library. A variegated library of CXCR4 or c-Kit variants can be produced by, for example, enzymatically lighting a mixture of synthetic oligonucleotides into gene sequences such that a degenerate set of potential CXCR4 or c-Kit sequences is expressible as individual polypeptides, or alternatively, as a set of larger fusion proteins (i.e., for phage display) containing the set of CXCR4 or c-Kit sequences therein. There are a variety of methods which can be used to produce libraries of potential CXCR4 or c-Kit homologues from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential CXCR4 or c-Kit sequences. Methods for synthesizing degenerate oligonucleotides are known in the art (see, i.e., Narang, S. A. (1983) Tetrahedron 39:3; Itakura et a. (1984) Annu. Rev. Biochem. 53:323; Itakura et al. (1984) Science 198:1056; Ike et al. (1983) Nucleic Acids Res. 11:477.

In addition, libraries of fragments of the CXCR4 or c-Kit protein coding can be used to generate a variegated population of CXCR4 or c-Kit fragments for screening and subsequent selection of homologues of a CXCR4 or c-Kit protein. In one embodiment, a library of coding sequence fragments can be generated by treating a double stranded PCR fragment of a CXCR4 or c-Kit coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the CXCR4 or c-Kit protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of CXCR4 or c-Kit homologues. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify CXCR4 homologues (Arkin and Youvan (1992) Proc. Natl. Acad. Sci. USA 89:7811-7815; Delagrave et al. (1993) Protein Eng. 6(3):327-331).

An isolated CXCR4 or c-Kit protein, or a portion or fragment thereof, can be used as an immunogen to generate antibodies that bind CXCR4 or c-Kit using standard techniques for polyclonal and monoclonal antibody preparation. The full-length CXCR4 or c-Kit protein can be used or, alternatively, the invention provides antigenic peptide fragments of CXCR4 or c-Kit for use as immunogens. The antigenic peptide of CXCR4 or c-Kit comprises at least 8 amino acid residues of the amino acid sequence shown in SEQ ID NO:2 or 4 or a homologous amino acid sequence as described herein and encompasses an epitope of CXCR4 or c-Kit such that an antibody raised against the peptide forms a specific immune complex with CXCR4 or c-Kit. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of CXCR4 or c-Kit that are located on the surface of the protein, i.e., hydrophilic regions.

A CXCR4 or c-Kit immunogen typically is used to prepare antibodies by immunizing a suitable subject, (i.e., rabbit, goat, mouse or other mammal) with the immunogen. An appropriate immunogenic preparation can contain, for example, recombinantly expressed CXCR4 or c-Kit protein or a chemically synthesized CXCR4 or c-Kit peptide. The preparation can further include an adjuvant, such as Freund's complete or incomplete adjuvant, or similar immunostimulatory agent. Immunization of a suitable subject with an immunogenic CXCR4 or c-Kit preparation induces a polyclonal anti-CXCR4 or c-Kit antibody response.

Accordingly, another aspect of the invention pertains to the use of anti-CXCR4 or c-Kit antibodies. The term “antibody” as used herein refers to immunoglobulin molecules and immunologically active portions of immunoglobulin molecules, i.e., molecules that contain an antigen binding site which specifically binds (immunoreacts with) an antigen, such as CXCR4 or c-Kit. Examples of immunologically active portions of immunoglobulin molecules include F(ab) and F(ab′)2 fragments which can be generated by treating the antibody with an enzyme such as pepsin. The invention provides polyclonal and monoclonal antibodies that bind CXCR4 or c-Kit. The term “monoclonal antibody” or “monoclonal antibody composition”, as used herein, refers to a population of antibody molecules that contain only one species of an antigen binding site capable of immunoreacting with a particular epitope of CXCR4 or c-Kit. A monoclonal antibody composition thus typically displays a single binding affinity for a particular CXCR4 or c-Kit protein with which it immunoreacts.

Polyclonal anti-CXCR4 or c-Kit antibodies can be prepared as described above by immunizing a suitable subject with a CXCR4 or c-Kit immunogen. The anti-CXCR4 or c-Kit antibody titer in the immunized subject can be monitored over time by standard techniques, such as with an enzyme linked immunosorbent assay (ELISA) using immobilized CXCR4 or c-Kit. If desired, the antibody molecules directed against CXCR4 or c-Kit can be isolated from the mammal (i.e., from the blood) and further purified by well known techniques, such as protein A chromatography to obtain the IgG fraction. At an appropriate time after immunization, i.e., when the anti-CXCR4 or c-Kit antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a CXCR4 or c-Kit immunogen as described above, and the culture supernatants of the resulting hybridoma cells are screened to identify a hybridoma producing a monoclonal antibody that binds CXCR4 or c-Kit.

Any of the many well known protocols used for fusing lymphocytes and immortalized cell lines can be applied for the purpose of generating an anti-CXCR4 or c-Kit monoclonal antibody (see, i.e., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (i.e., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, i.e., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind CXCR4 or c-Kit, i.e., using a standard ELISA assay.

Alternative to preparing monoclonal antibody-secreting hybridomas, a monoclonal anti-CXCR4 or c-Kit antibody can be identified and isolated by screening a recombinant combinatorial immunoglobulin library (i.e., an antibody phage display library) with CXCR4 or c-Kit to thereby isolate immunoglobulin library members that bind CXCR4 or c-Kit, respectively. Kits for generating and screening phage display libraries are commercially available (i.e., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Bio/Technology 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. Nature (1990) 348:552-554.

Additionally, recombinant anti-CXCR4 or c-Kit antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-CXCR4 or c-Kit antibody (i.e., monoclonal antibody) can be used to isolate CXCR4 or c-Kit by standard techniques, such as affinity chromatography or immunoprecipitation. An anti-CXCR4 or c-Kit antibody can facilitate the purification of natural CXCR4 or c-Kit from cells and of recombinantly produced CXCR4 or c-Kit expressed in host cells. Moreover, an anti-CXCR4 or c-Kit antibody can be used to detect CXCR4 or c-Kit protein (i.e., in a cellular lysate or cell supernatant) in order to evaluate the abundance and pattern of expression of the CXCR4 or c-Kit protein. Anti-CXCR4 or c-Kit antibodies can be used diagnostically to monitor protein levels in tissue as part of a clinical testing procedure, i.e., to, for example, determine the efficacy of a given treatment, regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

III. Recombinant Expression Vectors and Host Cells

Another aspect of the invention pertains to the use of vectors, preferably expression vectors, containing a nucleic acid encoding CXCR4, c-Kit, CXCR4 modulators, c-Kit modulators, or portions thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (i.e., bacterial vectors having a bacterial origin of replication and episomal mammalian vectors). Other vectors (i.e., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (i.e., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (i.e., in an in vitro transcription/translation system or in a host cell when the vector is introduced into the host cell). The term “regulatory sequence” is intended to include promoters, enhancers and other expression control elements (i.e., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (i.e., tissue-specific regulatory sequences). In a preferred embodiment, a lung specific promoter is used to direct expression of the nucleotide sequence in lung cells. It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (i.e., CXCR4 or c-Kit proteins, mutant forms of CXCR4 or c-Kit, fusion proteins, etc.).

The recombinant expression vectors of the invention can be designed for expression of CXCR4 or c-Kit in prokaryotic or eukaryotic cells. For example, CXCR4 or c-Kit can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:3140), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. In one embodiment, the coding sequence of the CXCR4 or c-Kit is cloned into a pGEX expression vector to create a vector encoding a fusion protein comprising, from the N-terminus to the C-terminus, GST-thrombin cleavage site-CXCR4 or c-Kit. The fusion protein can be purified by affinity chromatography using glutathione-agarose resin. Recombinant CXCR4 or c-Kit unfused to GST can be recovered by cleavage of the fusion protein with thrombin.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the CXCR4 or c-Kit expression vector is a yeast expression vector. Examples of vectors for expression in yeast S. cerivisae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.).

Alternatively, CXCR4 or c-Kit can be expressed in insect cells using baculovirus expression vectors. Baculovirus vectors available for expression of proteins in cultured insect cells (i.e., Sf9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (i.e., tissue-specific regulatory elements are used to express the nucleic acid). Tissue-specific regulatory elements are known in the art. Non-limiting examples of suitable tissue-specific promoters include the muscle specific casein kinase promoter, the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (i.e., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (i.e., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to CXCR4 or c-Kit mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, CXCR4 or c-Kit protein can be expressed in bacterial cells such as E. coli, insect cells, yeast or mammalian cells (such as muscle cells, Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (i.e., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (i.e., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding CXCR4 or c-Kit or can be introduced on a separate vector. Cells stably transfected with the introduced nucleic acid can be identified by drug selection (i.e., cells that have incorporated the selectable marker gene will survive, while the other cells die).

A host cell used in the methods of the invention, such as a prokaryotic or eukaryotic host cell in culture, can be used to produce (i.e., express) CXCR4 or c-Kit protein. Accordingly, the invention further provides methods for producing CXCR4 or c-Kit protein using the host cells of the invention. In one embodiment, the method comprises culturing the host cell of invention (into which a recombinant expression vector encoding CXCR4 or c-Kit has been introduced) in a suitable medium until CXCR4 or c-Kit is produced. In another embodiment, the method further comprises isolating CXCR4 or c-Kit from the medium or the host cell.

The host cells of the invention can also be used to produce nonhuman transgenic animals. The nonhuman transgenic animals (i.e., mice, rats, monkeys, horses, dogs, turkeys, fish, cows, pigs, sheep, goats, frogs, or chickens) can be used, for example, in screening assays designed to identify agents or compounds, i.e., drugs, pharmaceuticals, etc., which can be used to modulate SCLC proliferation and/or metastasis. For example, in one embodiment, a host cell of the invention is a fertilized oocyte or an embryonic stem cell into which CXCR4 or c-Kit coding sequences have been introduced. Such host cells can then be used to create non-human transgenic animals in which exogenous CXCR4 or c-Kit sequences have been introduced into their genome or homologous recombinant animals in which endogenous CXCR4 or c-Kit sequences have been altered. Such animals are useful for studying the function and/or activity of CXCR4 or c-Kit and for identifying and/or evaluating modulators of CXCR4 or c-Kit activity. As used herein, a “transgenic animal” is a nonhuman animal, preferably a mammal, more preferably a rodent such as a rat or mouse, in which one or more of the cells of the animal includes a transgene. Other examples of transgenic animals include nonhuman primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a nonhuman animal, preferably a mammal, more preferably a mouse, in which an endogenous CXCR4 or c-Kit gene has been altered by homologous recombination between the endogenous gene and an exogenous DNA molecule introduced into a cell of the animal, i.e., an embryonic cell of the animal, prior to development of the animal.

A transgenic animal of the invention can be created by introducing CXCR4 or c-Kit encoding nucleic acid into the male pronuclei of a fertilized oocyte, i.e., by microinjection, retroviral infection, and allowing the oocyte to develop in a pseudopregnant female foster animal. The human CXCR4 or c-Kit cDNA sequence can be introduced as a transgene into the genome of a nonhuman animal. Alternatively, a nonhuman homologue of the human CXCR4 gene (SEQ ID NO:1) or c-Kit gene (SEQ ID NO:3), such as a mouse CXCR4 or c-Kit gene, can used as a transgene. Intronic sequences and polyadenylation signals can also be included in the transgene to increase the efficiency of expression of the transgene. A tissue-specific regulatory sequence(s) can be operably linked to the CXCR4 or c-Kit transgene to direct expression of CXCR4 or c-Kit protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the CXCR4 or c-Kit transgene in its genome and/or expression of CXCR4 or c-Kit mRNA in tissues or cells of the animals. A transgenic founder animal can then be used to breed additional animals carrying the transgene. Moreover, transgenic animals carrying a transgene encoding CXCR4 or c-Kit can further be bred to other transgenic animals carrying other transgenes.

To create a homologous recombinant animal, a vector is prepared which contains at least a portion of a CXCR4 or c-Kit gene into which a deletion, addition or substitution has been introduced to thereby alter, i.e., functionally disrupt, the CXCR4 or c-Kit gene. The CXCR4 or c-Kit gene can be a human gene (i.e., from a human genomic clone isolated from a human genomic library screened with the cDNA of SEQ ID NO:1 or 3), but more preferably, is a nonhuman homologue of a human CXCR4 or c-Kit gene. For example, a mouse CXCR4 or c-Kit gene can be used to construct a homologous recombination vector suitable for altering an endogenous CXCR4 or c-Kit gene in the mouse genome. In a preferred embodiment, the vector is designed such that, upon homologous recombination, the endogenous CXCR4 or c-Kit gene is functionally disrupted (i.e., no longer encodes a functional protein; also referred to as a “knock out” vector). Alternatively, the vector can be designed such that, upon homologous recombination, the endogenous CXCR4 or c-Kit gene is mutated or otherwise altered but still encodes functional protein (i.e., the upstream regulatory region can be altered to thereby alter the expression of the endogenous CXCR4 or c-Kit protein). In the homologous recombination vector, the altered portion of the CXCR4 or c-Kit gene is flanked at its 5′ and 3′ ends by additional nucleic acid of the CXCR4 or c-Kit gene to allow for homologous recombination to occur between the exogenous CXCR4 or c-Kit gene carried by the vector and an endogenous CXCR4 or c-Kit gene in an embryonic stem cell. The additional flanking CXCR4 or c-Kit nucleic acid is of sufficient length for successful homologous recombination with the endogenous gene. Typically, several kilobases of flanking DNA (both at the 5′ and 3′ ends) are included in the vector (see i.e., Thomas, K. R. and Capecchi, M. R. (1987) Cell 51:503 for a description of homologous recombination vectors). The vector is introduced into an embryonic stem cell line (i.e., by electroporation) and cells in which the introduced CXCR4 or c-Kit gene has homologously recombined with the endogenous CXCR4 or c-Kit gene are selected (see i.e., Li, E. et al. (1992) Cell 69:915). The selected cells are then injected into a blastocyst of an animal (i.e., a mouse) to form aggregation chimeras (see i.e., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91101140 by Smithies et al.; WO 92/0968 by Zijlstra et al.; and WO 93/04169 by Berns et al.,

In one embodiment of the invention, transgenic animals are created using a vector containing a lung specific promoter operatively linked to a CXCR4 or c-Kit nucleic acid molecule.

In another embodiment, transgenic nonhuman animals can be produced which contain selected systems which allow for regulated expression of the transgene. One example of such a system is the cre/loxP recombinase system of bacteriophage P1. For a description of the cre/loxP recombinase system, see, i.e., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, ie., by mating two transgenic animals, one containing a transgene encoding a selected protein and the other containing a transgene encoding a recombinase.

Clones of the nonhuman transgenic animals described herein can also be produced according to the methods described in Wilmut, I. et al. (1997) Nature 385:810-813 and PCT International Publication Nos. WO 97/07668 and WO 97/07669. In brief, a cell, i.e., a somatic cell, from the transgenic animal can be isolated and induced to exit the growth cycle and enter Go phase. The quiescent cell can then be fused, i.e., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring borne of this female foster animal will be a clone of the animal from which the cell, i.e., the somatic cell, is isolated.

IV. Pharmaceutical Compositions

The CXCR4 and/or c-Kit modulators (also referred to herein as “active compounds”) of the invention can be incorporated into pharmaceutical compositions suitable for administration to a subject, i.e., a human. Such compositions typically comprise the nucleic acid molecule, protein, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral, i.e., intravenous, intradermal, subcutaneous, oral (Le., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (i.e., a CXCR4 modulator and/or a c-Kit modulator) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, i.e., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (i.e., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

V. Gene Therapy

In one embodiment of the invention, the nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see i.e., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, i.e. retroviral vectors, the pharmaceutical preparation can include one or more cells which produce the gene delivery system.

Viral vectors include, for example, recombinant retroviruses, adenovirus, adeno-associated virus, and herpes simplex virus-1. Retrovirus vectors and adeno-associated virus vectors are generally understood to be the recombinant gene delivery system of choice for the transfer of exogenous genes in vivo, particularly into humans. Adenovirus preferentially targets the liver when administered systemically (greater than 90+%; (Antinozzi et al. (1999) Annu. Rev. Nutr. 19:511-544) for reasons that may have to do with the expression of viral receptors or the lack of vascular barriers in the liver. Alternatively they can be used for introducing exogenous genes ex vivo into liver cells in culture. These vectors provide efficient delivery of genes into liver cells, and the transferred nucleic acids are stably integrated into the chromosomal DNA of the host cell.

A major prerequisite for the use of viruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol env) is replaced by a gene of interest rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines for preparing both ecotropic and amphotropic retroviral systems include ψCrip, ψCre, ψ2 and ψAm.

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. (1989) Proc. Natl. Acad. Sci. USA 86:9079-9083; Julan et al. (1992) J. Gen. Virol. 73:3251-3255; and Goud et al. (1983) Virology 163:251-254); or coupling cell surface receptor ligands to the viral env proteins (Neda et al. (1991) J. Biol. Chem. 266:14143-14146). Coupling can be in the form of the chemical cross-linking with a protein or other variety (i.e. lactose to convert the env protein to an asialoglycoprotein), as well as by generating fusion proteins (i.e. single-chain antibody/env fusion proteins). Thus, in a specific embodiment of the invention, viral particles containing a nucleic acid molecule containing a gene of interest operably linked to appropriate regulatory elements, are modified for example according to the methods described above, such that they can specifically target subsets of liver cells. For example, the viral particle can be coated with antibodies to surface molecule that are specific to certain types of liver cells. This method is particularly useful when only specific subsets of liver cells are desired to be transfected.

Another viral gene delivery system useful in the present invention ublizes adenovirus-derived vectors. The genome of an adenovirus can be manipulated such that it encodes and expresses a gene product of interest but is inactivated in terms of its ability to replicate in a normal lytic viral life cycle. See for example Berkner et al. (1988) Biotechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (i.e., Ad2, Ad3, Ad7 etc.) are well known to those skilled in the art. Recombinant adenoviruses can be advantageous in certain circumstances in that they are not capable of infecting nondividing cells. Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (i.e., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, i.e., Jones et al. (1979) Cell 16:683; Berkner et al., supra; and Graham et al. in Methods in Molecular Biology, E. J. Murray, Ed. (Humana, Clifton, N.J., 1991) vol. 7. pp. 109-127). Expression of the gene of interest comprised in the nucleic acid molecule can be under control of, for example, the E1A promoter, the major late promoter (MLP) and associated leader sequences, the E3 promoter, or exogenously added promoter sequences.

Yet another viral vector system useful for delivery of a nucleic acid molecule comprising a gene of interest is the adeno-associated virus (MV). Adeno-associated virus is a naturally occurring defective virus that requires another virus, such as an adenovirus or a herpes virus, as a helper virus for efficient replication and a productive life cycle. (For a review see Muzyczka et al. Curr. Topics Microbiol. Immunol. (1992) 158:97-129). Adeno-associated viruses exhibit a high frequency of stable integration (see for example Flotte et al. (1992) Am. J. Respir. Cell. Mol. Biol. 7:349-356; Samulski et al. (1989) J. Virol. 63:3822-3828; and McLaughlin et a. (1989) J. Virol. 62:1963-1973). Vectors containing as few as 300 base pairs of AAV can be packaged and can integrate. Space for exogenous DNA is limited to about 4.5 kb. An MV vector such as that described in Tratschin et al. (1985) Mol. Cell. Biol. 5:3251-3260 can be used to introduce DNA into T cells. A variety of nucleic acids have been introduced into different cell types using MV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790). Other viral vector systems that may have application in gene therapy have been derived from herpes virus, vaccinia virus, and several RNA viruses.

Still another viral vector system useful for delivery of a nucleic acid molecule comprising a gene of interest include the Herpes simplex virus type 1 (HSV-1) amplicon vectors for transfer of a gene into muscle (Wang, Y. et al. (2002) Hum. Gene. Ther. 13(2):261-273);

Other methods relating to the use of viral vectors in gene therapy can be found in, i.e., Kay, M. A. (1997) Chest 111(6 Supp.):138S-142S; Ferry, N. and Heard, J. M. (1998) Hum. Gene Ther. 9:1975-81; Shiratory, Y. et al. (1999) Liver 19:265-74; Oka, K. et al. (2000) Curr. Opin. Lipidol. 11:179-86; Thule, P. M. and Liu, J. M. (2000) Gene Ther. 7:1744-52; Yang, N. S. (1992) Crit. Rev. Biotechnol. 12:335-56; Alt, M. (1995) J. Hepatol. 23:746-58; Brody, S. L. and Crystal, R. G. (1994) Ann. N.Y. Acad. Sci. 716:90-101; Strayer, D. S. (1999) Expert Opin. Invetig. Drugs 8:2159-2172; Smith-Arica, J. R. and Bartlett, J. S. (2001) Curr. Cardiol. Rep. 3:43-49; and Lee, H. C. et al. (2000) Nature 408:483-8.

VI. Screening Assays

The nucleic acid molecules, polypeptides, polypeptide homologues, modulators, and antibodies described herein can be used in methods of treatment as well as drug screening assays. The invention provides a method (also referred to herein as a screening assay”) for identifying modulators, i.e., candidate or test compounds or agents (i.e., peptides, peptidomimetics, small molecules or other drugs) which bind to CXCR4 or c-Kit proteins, have an inhibitory effect on, for example, CXCR4 or c-Kit expression or CXCR4 or c-Kit activity, or have an inhibitory effect on, for example, the expression or activity of a CXCR4 or c-Kit target molecule. Such modulators of CXCR4 or c-Kit may be used in the methods of the invention to modulate cellular proliferation, movement, motility, adhesion, shape, morphological change, and/or metastasis of SCLC cells. Such modulators may be further used to treat a subject with SCLC.

In one embodiment, the invention provides assays for screening candidate or test compounds which are target molecules of a CXCR4 or c-Kit protein or polypeptide or biologically active portion thereof. In another embodiment, the invention provides assays for screening candidate or test compounds which bind to or modulate the activity of a CXCR4 or c-Kit protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:45).

Examples of methods for the synthesis of molecular libraries can be found in the art, for example, in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (i.e., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.).

In one embodiment, an assay is a cell-based assay in which a cell which expresses a CXCR4 or c-Kit protein or biologically active portion thereof is contacted with a test compound and the ability of the test compound to modulate CXCR4 or c-Kit activity is determined. Determining the ability of the test compound to modulate CXCR4 activity can be accomplished by monitoring, for example, SDF-1α induced PI3-K activity (e.g., Akt and/or p70 S6 kinase phosphorylation) or SDF-1α mediated cellular proliferation, adhesion, motility, or cell shape change in a cell which expresses CXCR4. The cell, for example, can be an SCLC cell, e.g., a primary tumor cell, or a cell from an SCLC tumor cell line.

The ability of the test compound to modulate CXCR4 or c-Kit binding to a target molecule can also be determined. Determining the ability of the test compound to modulate CXCR4 or c-Kit binding to a target molecule (e.g., SDF-1α or SCF, respectively) can be accomplished, for example, by coupling the CXCR4 or c-Kit target molecule with a radioisotope or enzymatic label such that binding of the CXCR4 or c-Kit target molecule to CXCR4 or c-Kit, respectively, can be determined by detecting the labeled CXCR4 or c-Kit target molecule in a complex. Alternatively, CXCR4 or c-Kit could be coupled with a radioisotope or enzymatic label to monitor the ability of a test compound to modulate CXCR4 or c-Kit binding to a CXCR4 or c-Kit target molecule in a complex. Determining the ability of the test compound to bind CXCR4 or c-Kit can be accomplished, for example, by coupling the compound with a radioisotope or enzymatic label such that binding of the compound to CXCR4 or c-Kit can be determined by detecting the labeled CXCR4 or c-Kit compound in a complex. For example, compounds (i.e., CXCR4 or c-Kit target molecules) can be labeled with 125I, 35S, 14C, or 3H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound or target molecule to interact with CXCR4 or c-Kit without the labeling of any of the interactants. For example, a microphysiometer can be used to detect the interaction of a compound with CXCR4 or c-Kit without the labeling of either the compound or the CXCR4 or c-Kit. McConnell, H. M. et al. (1992) Science 257:1906-1912. As used herein, a “microphysiometer” (i.e., Cytosensor) is an analytical instrument that measures the rate at which a cell acidifies its environment using a light-addressable potentiometric sensor (LAPS). Changes in this acidification rate can be used as an indicator of the interaction between a compound and CXCR4 or c-Kit.

In another embodiment, an assay is a cell-based assay comprising contacting a cell expressing a CXCR4 or c-Kit target molecule with a test compound and determining the ability of the test compound to modulate (Le. stimulate or inhibit) the activity of the CXCR4 or c-Kit target molecule. Determining the ability of the test compound to modulate the activity of a CXCR4 or c-Kit target molecule can be accomplished, for example, by determining the ability of a CXCR4 or c-Kit protein to bind to or interact with the CXCR4 or c-Kit target molecule, or by determining the ability of a CXCR4 or c-Kit protein to induce expression from a reporter construct.

Determining the ability of the CXCR4 or c-Kit protein, or a biologically active fragment thereof, to bind to or interact with a CXCR4 or c-Kit target molecule can be accomplished by one of the methods described above for determining direct binding. In a preferred embodiment, determining the ability of the CXCR4 or c-Kit protein to bind to or interact with a CXCR4 or c-Kit target molecule can be accomplished by determining the activity of the target molecule. For example, the activity of the target molecule can be determined by detecting induction of a cellular response (i.e., induction of PI3-K activity by SDF-1α or SCF), detecting catalytic/enzymatic activity of the target molecule upon an appropriate substrate, detecting the induction of a reporter gene (comprising a target-responsive regulatory element operatively linked to a nucleic acid encoding a detectable marker, i.e., luciferase), or detecting a target-regulated cellular response (i.e., proliferation).

In yet another embodiment, an assay of the present invention is a cell-free assay in which a CXCR4 or c-Kit protein or biologically active portion thereof is contacted with a test compound and the ability of the test compound to bind to the CXCR4 or c-Kit protein or biologically active portion thereof is determined. Preferred biologically active portions of the CXCR4 or c-Kit proteins to be used in assays of the present invention include fragments which participate in interactions with target molecules (e.g., SDF-1α or SCF). Binding of the test compound to the CXCR4 or c-Kit protein can be determined either directly or indirectly as described above. In a preferred embodiment, the assay includes contacting the CXCR4 or c-Kit protein or biologically active portion thereof with a known compound which binds CXCR4 or c-Kit to form an assay mixture, contacting the assay mixture with a test compound, and determining the ability of the test compound to interact with a CXCR4 or c-Kit protein, wherein determining the ability of the test compound to interact with a CXCR4 or c-Kit protein comprises determining the ability of the test compound to preferentially bind to CXCR4 or c-Kit or biologically active portion thereof as compared to the known compound.

In another embodiment, the assay is a cell-free assay in which a CXCR4 or c-Kit protein or biologically active portion thereof is contacted with a test compound and the ability of the test compound to modulate (i.e., stimulate or inhibit) the activity of the CXCR4 or c-Kit protein or biologically active portion thereof is determined. Determining the ability of the test compound to modulate the activity of a CXCR4 or c-Kit protein can be accomplished, for example, by determining the ability of the CXCR4 or c-Kit protein to bind to a CXCR4 or c-Kit target molecule by one of the methods described above for determining direct binding. Determining the ability of the CXCR4 or c-Kit protein to bind to a CXCR4 or c-Kit target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct Biol. 5:699-705. As used herein, “BIA” is a technology for studying biospecific interactions in real time, without labeling any of the interactants (i.e., BIAcore). Changes in the optical phenomenon of surface plasmon resonance (SPR) can be used as an indication of real-time reactions between biological molecules.

In an alternative embodiment, determining the ability of the test compound to modulate the activity of a CXCR4 or c-Kit protein can be accomplished by determining the ability of the CXCR4 or c-Kit protein to further modulate the activity of a downstream effector of a CXCR4 or c-Kit target molecule. For example, the activity of the effector molecule on an appropriate target can be determined or the binding of the effector to an appropriate target can be determined as previously described.

In yet another embodiment, the cell-free assay involves contacting a CXCR4 or c-Kit protein or biologically active portion thereof with a known compound which binds the CXCR4 or c-Kit protein (i.e., SDF-1α or SCF, respectively) to form an assay mixture, contacting the assay mixture with a test compound, and determining the ability of the test compound to interact with the CXCR4 protein, wherein determining the ability of the test compound to interact with the CXCR4 or c-Kit protein comprises determining the ability of the CXCR4 or c-Kit protein to preferentially bind to or modulate the activity of a CXCR4 or c-Kit target molecule.

In more than one embodiment of the above assay methods of the present invention, it may be desirable to immobilize either CXCR4 or c-Kit or its target molecule to facilitate separation of complexed from uncomplexed forms of one or both of the proteins, as well as to accommodate automation of the assay. Binding of a test compound to a CXCR4 or c-Kit protein, or interaction of a CXCR4 or c-Kit protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-5-transferase/CXCR4 or c-Kit fusion proteins or glutathione-5-transferase/target fusion proteins can be adsorbed onto glutathione sepharose beads (Sigma Chemical, St. Louis, Mo.) or glutathione derivatized micrometer plates, which are then combined with the test compound or the test compound and either the non-adsorbed target protein or CXCR4 or c-Kit protein, and the mixture incubated under conditions conducive to complex formation (i.e., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of CXCR4 or c-Kit binding or activity determined using standard techniques.

Other techniques for immobilizing proteins on matrices can also be used in the screening assays of the invention. For example, either a CXCR4 or c-Kit protein or a CXCR4 or c-Kit target molecule can be immobilized utilizing conjugation of biotin and streptavidin. Biotinylated CXCR4 or c-Kit protein or target molecules can be prepared from biotin-NHS(N-hydroxy-succinimide) using techniques known in the art (i.e., biotinylation kit, Pierce Chemicals, Rockford, Ill.), and immobilized in the wells of streptavidin-coated 96 well plates (Pierce Chemical). Alternatively, antibodies reactive with CXCR4 or c-Kit protein or target molecules but which do not interfere with binding of the CXCR4 or c-Kit protein to its target molecule can be derivatized to the wells of the plate, and unbound target or CXCR4 or c-Kit protein trapped in the wells by antibody conjugation. Methods for detecting such complexes, in addition to those described above for the GST-immobilized complexes, include immunodetection of complexes using antibodies reactive with the CXCR4 or c-Kit protein or target molecule, as well as enzyme-linked assays which rely on detecting an enzymatic activity associated with the CXCR4 or c-Kit protein or target molecule.

In another embodiment, modulators of CXCR4 or c-Kit expression are identified in a method wherein a cell is contacted with a candidate compound and the expression of CXCR4 or c-Kit mRNA or protein in the cell is determined. The level of expression of CXCR4 or c-Kit mRNA or protein in the presence of the candidate compound is compared to the level of expression of CXCR4 or c-Kit mRNA or protein in the absence of the candidate compound. The candidate compound can then be identified as a modulator of CXCR4 or c-Kit expression based on this comparison. For example, when expression of CXCR4 or c-Kit mRNA or protein is greater (statistically significantly greater) in the presence of the candidate compound than in its absence, the candidate compound is identified as a stimulator of CXCR4 or c-Kit mRNA or protein expression. Alternatively, when expression of CXCR4 or c-Kit mRNA or protein is less (statistically significantly less) in the presence of the candidate compound than in its absence, the candidate compound is identified as an inhibitor of CXCR4 or c-Kit mRNA or protein expression. The level of CXCR4 or c-Kit mRNA or protein expression in the cells can be determined by methods described herein for detecting CXCR4 or c-Kit mRNA or protein.

In yet another aspect of the invention, the CXCR4 or c-Kit proteins can be used as “bait proteins” in a two-hybrid assay or three-hybrid assay (see, i.e., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300) to identify other proteins which bind to or interact with CXCR4 or c-Kit (“CXCR4 or c-Kit-binding proteins” or “CXCR4 or c-Kit-bp”) and are involved in CXCR4 or c-Kit activity. Such CXCR4 or c-Kit-binding proteins are also likely to be involved in the propagation of signals by the CXCR4 or c-Kit proteins or CXCR4 or c-Kit targets as, for example, downstream elements of a CXCR4 or c-Kit-mediated signaling pathway. Alternatively, such CXCR4 or c-Kit-binding proteins may be CXCR4 or c-Kit inhibitors.

The two-hybrid system is based on the modular nature of most transcription factors, which consist of separable DNA-binding and activation domains. Briefly, the assay utilizes two different DNA constructs. In one construct, the gene that codes for a CXCR4 or c-Kit protein is fused to a gene encoding the DNA binding domain of a known transcription factor (i.e., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a CXCR4 or c-Kit dependent complex, the DNA-binding and activation domains of the transcription factor are brought into close proximity. This proximity allows transcription of a reporter gene (i.e., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the CXCR4 or c-Kit protein.

In another aspect, the invention pertains to a combination of two or more of the assays described herein. For example, a modulating agent can be identified using a cell-based or a cell-free assay, and the ability of the agent to modulate the activity of a CXCR4 or c-Kit protein can be confirmed in vivo, i.e., in an animal such as a mouse model for SCLC.

This invention further pertains to novel agents identified by the above-described screening assays. Accordingly, it is within the scope of this invention to further use an agent identified as described herein in an appropriate animal model. For example, an agent identified as described herein (i.e., a CXCR4 or c-Kit modulating agent, an antisense CXCR4 or c-Kit nucleic acid molecule, a CXCR4 or c-Kit specific antibody, or a CXCR4 or c-Kit binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

In yet another embodiment, the invention provides a method for identifying a compound (i.e., a screening assay) capable of use in the treatment of SCLC. This method typically includes the step of assaying the ability of the compound or agent to modulate the expression of the CXCR4 or c-Kit nucleic acid or the activity of the CXCR4 or c-Kit protein thereby identifying a compound for treating SCLC. Methods for assaying the ability of the compound or agent to modulate the expression of the CXCR4 or c-Kit nucleic acid or activity of the CXCR4 or c-Kit protein are typically cell-based assays. For example, cells which are sensitive to ligands which transduce signals via a pathway involving CXCR4 or c-Kit can be induced to overexpress a CXCR4 or c-Kit protein in the presence and absence of a candidate compound. Candidate compounds which produce a statistically significant change in CXCR4 or c-Kit dependent responses (either stimulation or inhibition) can be identified. In one embodiment, expression of the CXCR4 or c-Kit nucleic acid or activity of a CXCR4 or c-Kit protein is modulated in cells and the effects of candidate compounds on the readout of interest (such as rate of cell proliferation or differentiation) are measured. For example, the expression or activity of genes which are up- or down-regulated in response to a CXCR4 protein-dependent signal cascade can be assayed (e.g., the phosphorylation of Akt and/or p70 S6 kinase). In preferred embodiments, the regulatory regions of such genes, i.e., the 5′ flanking promoter and enhancer regions, are operably linked to a detectable marker (such as luciferase) which encodes a gene product that can be readily detected. Phosphorylation of CXCR4 or c-Kit or CXCR4 or c-Kit target molecules can also be measured, for example, by immunoblotting.

Alternatively, modulators of CXCR4 or c-Kit nucleic acid expression (i.e., compounds which can be used to treat SCLC) can be identified in a method wherein a cell is contacted with a candidate compound and the expression of CXCR4 or c-Kit mRNA or protein in the cell is determined. The level of expression of CXCR4 or c-Kit mRNA or protein in the presence of the candidate compound is compared to the level of expression of CXCR4 or c-Kit mRNA or protein in the absence of the candidate compound. The candidate compound can then be identified as a modulator of CXCR4 or c-Kit nucleic acid expression based on this comparison and be used to treat a disorder characterized by aberrant CXCR4 or c-Kit nucleic acid expression. For example, when expression of CXCR4 or c-Kit mRNA or polypeptide is greater (statistically significantly greater) in the presence of the candidate compound than in its absence, the candidate compound is identified as a stimulator of CXCR4 or c-Kit nucleic acid expression. Alternatively, when CXCR4 or c-Kit nucleic acid expression is less (statistically significantly less) in the presence of the candidate compound than in its absence, the candidate compound is identified as an inhibitor of CXCR4 or c-Kit nucleic acid expression. The level of CXCR4 or c-Kit nucleic acid expression in the cells can be determined by methods described herein for detecting CXCR4 or c-Kit mRNA or protein.

Modulators of CXCR4 or c-Kit protein activity and/or CXCR4 or c-Kit nucleic acid expression identified according to these drug screening assays can be used to treat SCLC. Modulators of CXCR4 or c-Kit protein activity and/or CXCR4 or c-Kit nucleic acid expression may also be used to treat disorders related to other functions of CXCR4 or c-Kit unrelated to SCLC (e.g., AIDS). These methods of treatment include the steps of administering the modulators of CXCR4 or c-Kit protein activity and/or nucleic acid expression, i.e., in a pharmaceutical composition as described in subsection IV above, to a subject in need of such treatment, ie., a subject with a disorder described herein.

VII. Monitoring the Effectiveness of an Anti-SCLC Agent

The presence or activity level of CXCR4 and/or c-Kit may be used to: 1) determine if SCLC can be or is likely to be successfully treated by an agent or combination of agents; 2) determine if SCLC is responding to treatment with an agent or combination of agents; 3) select an appropriate agent or combination of agents for treating SCLC; 4) monitor the effectiveness of an ongoing treatment; and/or 5) identify new treatments (either single agent or combination of agents). In particular, CXCR4 and/or c-Kit may be utilized as markers (surrogate and/or direct) to determine appropriate therapy, to monitor clinical therapy and human trials of a drug being tested for efficacy, and to develop new agents and therapeutic combinations.

Accordingly, the present invention provides methods for determining whether an agent, e.g., a chemotherapeutic agent, can be used to inhibit proliferation or metastasis of SCLC comprising the steps of:
a) obtaining a sample of lung cells;
b) determining whether the cells express CXCR4;
   thereby determining that the agent can be used to inhibit proliferation or metastasis of small cell lung cancer when CXCR4 is expressed. In a further embodiment, the method comprises determining whether the cells express c-Kit, thereby determining that the agent can be used to inhibit proliferation or metastasis of small cell lung cancer when CXCR4 and c-Kit are expressed.
   In another embodiment, the invention provides a method for determining whether a patient would benefit from treatment with an agent that inhibits CXCR4 comprising:
a) obtaining a lung sample from the patient; and
b) determining whether CXCR4 is expressed in the sample. In another embodiment, the invention provides a method for determining whether a patient would benefit from treatment with an agent that inhibits CXCR4 and an agent that inhibits c-Kit comprising:
a) obtaining a lung sample from the patient;
b) determining whether CXCR4 is expressed in the sample; and
c) determining whether c-Kit is expressed in the sample.
   The activity level of CXCR4 and/or c-Kit can also be used to assess whether SCLC has become refractory to an ongoing treatment (e.g., a chemotherapeutic treatment). When SCLC is no longer responding to a treatment the activity profile of the cells will change: the level of activity of CXCR4 and/or c-Kit will be reduced. Accordingly, in another embodiment, the invention provides a method for determining whether treatment with a CXCR4 inhibitor should be continued or discontinued in an SCLC patient, comprising:
a) obtaining two or more samples comprising cells from a patient during the course of treatment;
b) determining the level of activity in the cells of CXCR4; and
c) continuing treatment when the activity of CXCR4 does not increase during treatment In another embodiment, the invention provides a method for determining whether treatment with a combination of a CXCR4 inhibitor and a receptor tyrosine kinase inhibitor should be continued or discontinued in a small cell lung cancer patient, comprising:
a) obtaining two or more samples comprising cells from a patient during the course of treatment;
b) determining the level of activity in the cells of CXCR4;
c) determining the level of activity in the cells of c-Kit; and
d) continuing treatment when the activity levels of CXCR4 and/or c-Kit do not increase during treatment.

This embodiment of the present invention relies on comparing two or more samples obtained from a patient undergoing anti-SCLC treatment. It will be appreciated that the samples may be from the lung of the patent (e.g. SCLC cells, tumor cells, etc.), obtained using standard methods. In general, it is preferable to obtain a first sample from the patient prior to beginning therapy and one or more samples during treatment. In such a use, a baseline of activity prior to therapy is determined and then changes in the baseline state of expression is monitored during the course of therapy. Alternatively, two or more successive samples obtained during treatment can be used without the need of a pre-treatment baseline sample. In such a use, the first sample obtained from the subject is used as a baseline for determining whether the activity of CXCR4 and/or c-Kit is increasing or decreasing.

In general, when monitoring the effectiveness of a therapeutic treatment, two or more samples from the patient are examined. Preferably, three or more successively obtained samples are used, including at least one pretreatment sample.

In one embodiment of the invention, the expression of CXCR4 and/or c-Kit is detected by measuring CXCR4 and/or c-Kit mRNA levels, respectively. In yet another embodiment of the invention, the expression of CXCR4 and/or c-Kit is detected by measuring CXCR4 and/or c-Kit protein levels, respectively.

As used herein, the term “agent” is defined broadly as anything that SCLC cells, may be exposed to in a therapeutic protocol, preferably an agent that has been identified as being effective for the treatment of SCLC, e.g., using the methods described herein. In the context of the present invention, such agents include, but are not limited to, chemotherapeutic agents, radiation and ultraviolet light. In a preferred embodiment, the agent is a CXCR4 inhibitor (e.g., AMD-3100, ALX40-4C, T22, T140, Met-SDF-1beta, T134, AMD-3465, or an agent identified by the screening methods described herein). In another embodiment, the agent is a receptor tyrosine kinase inhibitor, e.g., a c-Kit inhibitor. In a preferred embodiment, the c-Kit inhibitor is imatinib mesylate (STI571 or Gleevec™). In a further preferred embodiment, the agent is a combination of a CXCR4 inhibitor and a receptor tyrosine kinase inhibitor.

Further to the above, the language “chemotherapeutic agent” is intended to include chemical reagents which inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990)), and are typically used to treat neoplastic diseases. In a preferred embodiment, the chemotherapeutic agents used in the methods of the invention are chemotherapeutic agents used to treat SCLC.

The methods of the present invention may also be used to modulate the proliferation, growth, movement, motility, adhesion, morphology, and/or metastasis of any type of cancer or tumor cells that express CXCR4 and/or c-Kit, e.g., SCLC cells. As used herein, cancer cells, including tumor cells, refer to cells that divide at an abnormal (increased) rate.

The source of the lung or SCLC cells used in the present method will be based on how the method of the present invention is being used. For example, if the method is being used to determine whether a patient's SCLC can be treated with an agent, or a combination of agents, then the preferred source of cells will be lung cancer cells obtained from lung biopsy from the patient, e.g., a tumor biopsy. If the method is being used to monitor the effectiveness of a therapeutic protocol, then a lung tissue sample from the patient being treated is the preferred source. If the method is being used to identify new therapeutic agents or combinations, any SCLC cells, e.g., primary SCLC cells or an SCLC cell line, can be used.

A skilled artisan can readily select and obtain the appropriate cells that are used in the present method. For cancer cell lines, sources such as The National Cancer Institute, for the NCI-H69 cells, are preferred. For cancer cells obtained from a patient, standard biopsy methods can be employed.

In the methods of the present invention, the expression or level of activity of CXCR4 and/or c-Kit is determined. As used herein, the expression of CXCR4 or c-Kit refers to whether or not CXCR4 or c-Kit mRNA is expressed in the cells, e.g., the SCLC cells. As used herein, the level of activity of CXCR4 refers to, for example, the ability of SDF-1α to modulate proliferation, adhesion, motility, cell shape, or PI3-K activity in the cells. As used herein, the level of activity of c-Kit refers to, for example, the ability of SCF to modulate proliferation, adhesion, motility, cell shape, or PI3-K activity in the cells.

As used herein, a patient refers to any subject undergoing treatment for lung cancer (e.g., SCLC). The preferred subject will be a human patient undergoing chemotherapy treatment.

An exemplary method for detecting the presence or absence of a polypeptide or nucleic acid corresponding to CXCR4 and/or c-Kit in a biological sample involves obtaining a biological sample (e.g., a lung sample or a lung tumor sample) from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. For example, in vitro techniques for detection of mRNA include Northern hybridizations and in situ hybridizations. In vitro techniques for detection of a polypeptide corresponding to CXCR4 or c-Kit include enzyme linked immunosorbent assays (ELISAs), Western blots, immunoprecipitations and immunofluorescence. In vivo techniques for detection of genomic DNA include Southern hybridizations. Furthermore, in vivo techniques for detection of a polypeptide corresponding to CXCR4 or c-Kit include introducing into a subject a labeled antibody directed against the polypeptide. For example, the antibody can be labeled with a radioactive marker whose presence and location in a subject can be detected by standard imaging techniques.

A general principle of such diagnostic and prognostic assays involves preparing a sample or reaction mixture that may contain CXCR4 and/or c-Kit, and a probe, under appropriate conditions and for a time sufficient to allow CXCR4 and/or c-Kit and the probe to interact and bind, thus forming a complex that can be removed and/or detected in the reaction mixture. These assays can be conducted in a variety of ways.

For example, one method to conduct such an assay would involve anchoring CXCR4 and/or c-Kit or the probe onto a solid phase support, also referred to as a substrate, and detecting target CXCR4 and/or c-Kit probe complexes anchored on the solid phase at the end of the reaction. In one embodiment of such a method, a sample from a subject, which is to be assayed for presence of CXCR4 and/or c-Kit, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, CXCR4 and/or c-Kit or probe molecules which are immobilized through conjugation of biotin and streptavidin. Such biotinylated assay components can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the CXCR4 and/or c-Kit or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of CXCR4 and/or c-Kit/probe complexes anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect CXCR4 and/or c-Kit/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize CXCR4 and/or c-Kit can be accomplished without labeling either assay component (probe or marker) by utilizing a technology such as real-time Biomolecular Interaction Analysis (BIA).

Alternatively, in another embodiment, analogous diagnostic and prognostic assays can be conducted with CXCR4 and/or c-Kit and probe as solutes in a liquid phase. In such an assay, the complexed CXCR4 and/or c-Kit and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G. and Minton, A. P. (1993) Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the CXCR4 and/or c-Kit/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of mRNA corresponding to CXCR4 and/or c-Kit can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. In a preferred embodiment, a biological sample is preferably a lung sample or a lung tumor sample. Many expression detection methods use isolated RNA For in vitro methods, any RNA isolation technique that does not select against the isolation of mRNA can be utilized for the purification of RNA from ovarian cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question (e.g., CXCR4 and/or c-Kit) is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of CXCR4 and/or c-Kit mRNA.

An alternative method for determining the level of mRNA corresponding to CXCR4 and/or c-Kit in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the lung cells prior to detection. In such methods, a lung tumor or tissue sample is prepared/processed using known histological methods. The sample is then immobilized on a support, typically a glass slide, and then contacted with a probe that can hybridize to mRNA that encodes CXCR4 and/or c-Kit.

In another embodiment of the present invention, a polypeptide corresponding to CXCR4 and/or c-Kit is detected. A preferred agent for detecting a polypeptide of the invention is an antibody or antibody fragment capable of binding to CXCR4 and/or c-Kit, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

A variety of formats can be employed to determine whether a sample contains a protein (e.g., CXCR4 and/or c-Kit) that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether lung cells express CXCR4 and/or c-Kit.

In one format, antibodies, or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from lung cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

This invention is further illustrated by the following examples which should not be construed as limiting.

### EXAMPLES

Materials and Methods

Cell Lines and Cell Culture

Ten small cell lung cancer (SCLC) cell lines (NCI-H69, NCI-H82, NCI-H128, NCI-H146, NCI-H209, NCI-H249, NCI-H345, NCI-H446, NCI-H510, and NCI-H526) were purchased from the American Type Culture Collection (Rockville, Md.) and maintained in RPMI 1640 medium (Cellgro) supplemented with 10% (v/v) fetal calf serum (FCS). MO7e cells were maintained as described in Sattler, M. et al. (1997) J. Biol. Chem. 272:10248-53. All cell lines were incubated at 37° C. in a 5% CO₂ humidified atmosphere and harvested during log phase growth. Cells were deprived of growth factors by incubation in RPMI 1640 medium containing 0.5% (w/v) bovine serum albumin (BSA) (Sigma, St. Louis, Mo.) for 18 hours. In some experiments, cells were treated with 5 μM STI571 (Gleevec™, Novartis Pharmaceuticals, Basel, Switzerland) or 25 μM LY294002 (Sigma). Recombinant human SDF-1α and SCF (BioSource International, Inc., Camarillo, Calif.) were used in the conditions indicated in each experiment.

Cell Viability Assay

NCI-H69 cells (1×10⁶/ml) were cultured in serum-free (0.5% BSA) or serum-containing (0.5, 1, 5, or 10% FCS) media with or without 100 ng/ml SCF and/or SDF-1α. Viable cells were counted by trypan blue dye exclusion test every 24 hours up to 72 hours. Data was plotted as the mean±SD from three independent experiments. Student's t test was used for the statistical analysis of viable cell number under individual conditions. Differences were considered statistically significant at p<0.05.

RNase Protection Assay (RPA)

Chemokine receptor mRNA was detected using the hCR-6 multi-probe template set (RiboQuant, PharMingen, San Diego, Calif.) according to the manufacturer's protocol. This set contains DNA templates for CXCR-1, -2, -3, -4, Burkitt's lymphoma receptor (BLR)-1/CXCR5, BLR-2/CCR7, V28/CX3CR1, as well as ribosomal protein L32 and GAPDH (glyceradehyde-3-phosphate dehydrogenase) as controls. In brief, anti-sense RNA probes were generated from DNA templates by T7 RNA polymerase in the presence of [α-³²P] UTP (3,000 Ci/mmol; Life Science Products, Inc., Boston, Mass.). Labeled probes were hybridized overnight at 56° C. with 20 μg total RNA isolated using RNeasy kit (Qiagen, Hilden, Germany). As the negative and positive controls, 2 μg yeast tRNA and human control RNA-2 were used, respectively. Unhybridized RNA was digested with RNase A and T1. RNase-protected probes were resolved on a denaturing 5% acrylamide-urea sequencing gel and identified by autoradiography.

FACS Analysis

Cells (1×10⁵) were washed three times in phosphate buffered saline (PBS) containing 0.5% BSA (PBS buffer), then incubated for 30 minutes at 4° C. with 10 μg/ml phycoerythrin (PE)-conjugated mouse-anti-human CXCR4 monoclonal antibody or PE-labeled mouse control IgG₂₈ (R&D Systems Inc., Minneapolis, Minn.). After washing the cells twice with PBS buffer to remove unbound antibodies, the stained cells were resuspended in 300 μl of PBS and analyzed by FACScan using Cell Quest software (Becton Dickinson Labware, Franklin Lakes, N.J.).

Adhesion Assay

The wells of a 96-well tissue culture plate (Corning-Costar, Cambridge, Mass.) pre-coated with 10 μg/ml human plasma fibronectin (FN) or human collagen type IV (col. IV) (Gibco BRL, Rockville, Md.) overnight at 4° C. were washed with PBS twice and blocked for 1 hour at 37° C. with RPMI 1640 medium containing 0.2% BSA (adhesion media) before plating cells. NCI-H446 cells (3×10⁵) were washed twice, resuspended in the adhesion media with or without SDF-1α (100 ng/ml), and plated onto uncoated, FN-, or col. IV-coated wells. Unattached cells were removed after incubation for 2 hours at 37° C. by gentle washing with adhesion media. The relative number of attached viable cells was determined by the MTT colorimetric assay (Sigma) following the instruction manual. Student's t test was used for the statistical analysis of the attached cell number and differences were taken significant at p<0.05.

Phase Contrast Microscopy for Cell Morphology

Serum-starved NCI-H69 cells (1×10⁶) were placed onto 6 well tissue culture plates (Becton Dickinson Labware) and incubated in serum free media in the absence or presence of STI571 (5 μM), SDF-1α (100 ng/ml) and/or SCF (100 ng/ml). Phase-contrast photomicrographs were taken at various time points (0, 1, 2, 4, 8, and 24 hours). For LY294002 studies, starved NCI-H446 cells (1×10⁶) were resuspended in serum free media and placed onto a 35 mm tissue culture dish (Becton Dickinson Labware). After 24 hours incubation in the absence or presence of LY294002 (25 μM) and/or SDF-1α (100 ng/ml), phase-contrast pictures were taken.

Analysis of Cell Motility by Time-Lapse Video Microscopy (TLVM)

NCI-H446 cells were plated as above and the dish was placed into a temperature controlled chamber at 37° C. in an atmosphere of 5% CO₂. The cells were examined by TLVM using an Olympus IX70 inverted microscope, Omega temperature controlled device, DVC1310 digital video camera, and QED Camera with Standalone 145 software. SDF-1α (100 ng/ml) was added into the culture after 6 hours and images were recorded for another 10 hours. Digital video images were saved every 90 seconds, and cell movement or morphological changes were analyzed with the NIH Image Analysis program. For movement analysis, the position of cell centroid was measured every 15 minutes and plotted to show the trace of centroid movement. The distance that the cell centroid transversed for each 90 seconds was calculated to determine the speed of the movement. For morphology analysis, the cell surface area and perimeter were measured to represent the degree of rugged shape. The frequency and period of formation and retraction of filopodia and uropods were also analyzed.

Immunoblotting

Cells were lysed in lysis buffer (20 mM Tris pH 8.0, 150 mM NaCl, 10% glycerol, 1% NP-40, 0.42% NaF) containing protease inhibitors (1 mM PMSF, 1 mM Na₃VO₄, 5 μg/ml aprotinin, 5 μg/ml leupeptin). Cell lysates were separated by 7.5% SDS-PAGE under reducing conditions and transferred to nitrocellulose membranes (Schleicher & Schuell, Dassel, Germany). Proteins were detected by immunoblotting using an enhanced chemiluminescence technique (NEN Life Science Products, Mass.). Rabbit polyclonal antibodies against c-Kit (C-19; Santa Cruz Biotechnology, Inc., Santa Cruz, Calif.), Akt, Akt (pSer 473), Akt (pThr 308), p70 S6 kinase, and p70 S6 kinase (pThr 389) (Cell Signaling Technology, Inc., Beverly, Mass.) and monoclonal antibodies against □-actin (AC-15; Sigma, St. Louis, Mo.) and phosphotyrosine (4G10, UBI, Lake Placid, N.Y.) were used.

### Example 1

### CXCR4 is Ubiquitously Expressed and c-kit is Variably Expressed in SCLC Cell Lines

The expression of chemokine receptor mRNAs in SCLC cells was determined by RPA. Yeast tRNA and human control RNA-2 were used as the negative and positive controls for CXCR4, respectively. All of the SCLC cell lines (10 total) tested expressed CXCR4 mRNA at various levels, with no detectable mRNA for other chemokine receptors. The human megakaryoblastic cell line MO7e, as a control, expressed CXCR3 as well as CXCR4. Expression of CXCR4 protein was confirmed by flow cytometric analysis in these SCLC cell lines and MO7e. The expression of CXCR4 was highest in NCI-H209 and NCI-H446 cells. The expression of c-Kit in these cell lines was evaluated by immunoblotting. 6 out of 10 SCLC cell lines tested expressed variable levels of c-Kit (lines H69, H128, H209, H345, H510, and H526), with high expression in the MO7e control cells. The blots were stripped and reprobed with an antibody against β-actin.

### Example 2

### SCF and SDF-1α Induce Proliferation of NCI-H69 Cells

The effect of SCF and SDF-1α on viability in NCI-H69 cells was analyzed. As described above in Example 1, NCI-H69 cells express both c-Kit and CXCR4, and thus these cells were used for many of the experiments. Without serum, neither SCF nor SDF-1α showed any effect on cell survival. On the other hand, in media containing 10% FCS, cell proliferation was significantly induced by SCF (21.5%, p=0.0373) and SDF-1α (26.6%, p=0.0133) separately or in combination (26.6%, p=0.0133) at 48 hours, as compared with untreated control. SCF and SDF-1α also conferred an increase in viable cell number at 72 hours (16.5%, p=0.0164 and 15.5%, p=0.0184, respectively, and 20.0%, p=0.0322, combined). Even though SCF and SDF-1α individually induced proliferation of NCI-H69 cells, there was no additive or synergistic effect seen with both the cytokine and chemokine combined. Similar results were observed for different concentrations of FCS tested (0.5, 1, and 5% FCS), implicating the importance of SCF and SDF-1α in proliferation of NCI-H69 SCLC cells.

### Example 3

### SDF-1α Regulates Adhesion, Motility, and Cell Shape in NCI-H446 SCLC Cells

Cytoskeletal functions such as increased cell motility, adhesion to extra cellular matrix (ECM) proteins, morphological change, and movement, are crucial for cancer cells to metastasize. To determine the effect of SDF-1α on cell motility and adhesion, NCI-H446 cells that express high amounts of CXCR4 and grow in an anchorage dependent fashion were used. In an adhesion assay, FN (3.84-fold, p=0.0002) and col. IV (2.98-fold, p=0.0124) were found to increase the adhesion of NCI-H446 cells as compared to the uncoated surface. In conjunction, SDF-1α stimulation further increased the attachment 3.14-fold on the uncoated surface (p<0.0001), but did not significantly enhance FN- and col. IV-mediated adhesion. In addition to cell adhesion, SDF-1α also markedly increased the motility and speed of NCI-H446 cells. The speed of cell movement was calculated using the distance that each cell centroid traversed per each 90 seconds. Morphological changes from round to polygonal shape including formation of neurite like projections, increased membrane ruffling, and more frequent filopodia and uropods formations were observed in response to SDF-1α. Filopodia formation in the presence of SDF-1α occurred much more frequently (13.14 vs. 2.86 times/hour/cell) and for longer periods of time (6.09 vs. 3.75 minutes/filopodium). Uropod formation was observed in 4 out of 7 (57.1%) SDF-1α treated cells. However, only 1 out of 7 (14.3%) untreated cells showed uropods. The period of time each uropod lasted also became much longer when induced by SDF-1α stimulation (12.2 vs. 5.0 minutes/uropod).

### Example 4

### PI3-K Regulates SDF-1α Induced Cell Motility of NCI-H446 SCLC Cells

This example describes experiments conducted to determine whether the PI3-K inhibitor LY294002 can inhibit SDF-1α induced cell motility in NCI-H446 SCLC cells. NCI-H446 cells were either left untreated or treated with SDF-1α (100 ng/ml) in the absence or presence of LY294002 (25 μM). Phase-contrast microscopy pictures were taken at the 24 hour time point. Most of the untreated cells kept their rounded shape and formed clusters, and nearly half of them attached weakly to the bottom of the dish. With SDF-1α, almost all cells tightly adhered to the bottom of the dish and neurite like projections were induced in many cells. In the presence of LY294002, more than 90% of the cells were floating and had rounded shapes in spite of SDF-1α treatment, although they could form clumps.

### Example 5

### CXCR4 and c-KIT Cooperatively Induce Morphological Changes in NCI-H69 SCLC Cells

It has not been shown previously that there is a functional interaction between CXCR4 and c-Kit in SCLC. NCI-H69 cells positive for both CXCR4 and c-Kit expression were either untreated or treated with SDF-1α (100 ng/ml) and/or SCF (100 ng/ml), in the absence or presence of STI571 (5 μM). Phase-contrast microscopy pictures were then taken after 8 hours. Morphological changes began to be apparent at 4 hours, and the changes plateaued between 8 and 24 hours. Neurite like actin formations were observed in response to SCF, and this morphological change was more apparent as projections when the cells were treated with SDF-1α. Neurite like projections induced by SDF-1α alone still formed even in the presence of STI571. STI571 treated NCI-H69 cells stimulated with both SDF-1α and SCF could not form any neurite like structures. STI571 inhibited the morphological changes only in the presence of SCF. These results suggest that there are important interactions between CXCR4 and c-Kit in SCLC that influence cell motility. Inhibition of the active c-Kit receptor by STI571 may lead to the inhibition of other active receptors such as CXCR4.

### Example 6

### SDF-1α and SCF Independently Regulate Phosphorylation of AKT and P70 S6 Kinase

PI3-K is important in the regulation of cytoskeletal functions in SDF-1α and SCF signaling (Ganju, R. K. et al. (1998) J. Biol. Chem. 273:23169-75; Linnekin, D. et al. (1999) Int. J. Biochem. Cell Biol. 31:1053-74; Vicente-Manzanares, M. et al. (1999) J. Immunol. 163:4001-12; Wang, J. F. et al. (2000) Blood 95:2505-13; Zhang, X. F. et al. (2001) Blood 97:3342-8). The activity of the PI3-K downstream targets Akt and p70 S6 kinase is regulated through critical serine/threonine (Ser/The) residues (Franke, T. F. et al. (1997) Cell 88:435-7; Pullen, N. and Thomas, G. (1997) FEBS Lett 410:78-82). Serum starved NCI-H69 cells were stimulated with SDF-1α (50 ng/ml) or SCF (50 ng/ml) for 0-60 minutes before lysis. Cell lysates were applied to a 7.5% SDS-PAGE gel and transferred to nitrocellulose membrane. The membrane was probed with monoclonal antibodies against phosphotyrosine (4G10) and β-actin and polyclonal antibodies against Akt (pSer 473) and p70 S6 kinase (S6K) (pThr 389). Both SDF-1α and SCF induced time dependent tyrosine phosphorylation of cellular proteins and Ser/Thr phosphorylation of Akt and p70 S6 kinase in NCI-H69 cells. Several tyrosine phosphorylated bands were identified between 70-120 kDa within 15 minutes of SDF-1α stimulation. On the other hand, maximal tyrosine phosphorylation of proteins at 60-90 kDa and 110-145 kDa occurred within 2.5-7.5 minutes in response to SCF. Dose-response studies indicated that optimal phosphorylation of cellular proteins was obtained with at least 25 ng/ml SDF-1α and 10 ng/ml SCF. Phosphorylation of Akt (Ser 473) and p70 S6 kinase (Thr 389) occurred in response to SDF-1α within 5 minutes and in response to SCF within 2.5 minutes.

### Example 7

### STI571 and LY294002 Inhibit Signal Transduction of CXCR4 and c-Kit Pathways

The small molecular inhibitors STI571 (which targets c-Kit) and LY294002 (which targets PI3-K) were utilized to determine the effects on downstream signaling by SDF-1α and SCF in NCI-H69 cells. Cells were left untreated or pre-treated with STI571 (5 μM) or LY294002 (25 μM) overnight in serum starved media and subsequently stimulated with 50 ng/ml SCF and/or 50 ng/ml SDF-1α for 15 minutes before lysis. Lysates were processed as described above in Example 6, and the membrane was probed with the phospho-specific or regular antibodies against Akt and p70 S6 kinase, as well as with an anti-c-Kit antibody. Cooperative phosphorylation of Akt at Ser 473/Thr 308 and p70 S6 kinase at Thr 389 was induced by SCF and SDF-1α. STI571 pre-treatment inhibited SCF but not SDF-1α induced phosphorylation. The expression levels of Akt, p70 S6 kinase, and c-Kit were not affected by any of these treatments. In contrast, LY294002 pre-treatment blocked SDF-1α as well as SCF induced phosphorylation of Akt and p70 S6 kinase.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of inhibiting cellular proliferation in a small cell lung cancer cell population comprising contacting the population with a CXCR4 inhibitor.

2. A method of inhibiting cellular movement or motility in a small cell lung cancer cell population comprising contacting the population with a CXCR4 inhibitor.

3. A method of modulating cellular adhesion in a small cell lung cancer cell population comprising contacting the population with a CXCR4 inhibitor.

4. A method of inhibiting morphological change in a small cell lung cancer cell population comprising contacting the population with a CXCR4 inhibitor.

5. The method of claim 1, further comprising contacting the small cell lung cancer cell population with a receptor tyrosine kinase inhibitor.

6. The method of claim 5, wherein the receptor tyrosine kinase inhibitor is a c-Kit inhibitor.

7. A method of treating a subject with small cell lung cancer comprising administering a CXCR4 inhibitor to the subject.

8. The method of claim 7, further comprising administering a receptor tyrosine kinase inhibitor to the subject.

9. The method of claim 8, wherein the receptor tyrosine kinase inhibitor is a c-Kit inhibitor.

10. A method of inhibiting metastasis of small cell lung cancer in a subject comprising administering a CXCR4 inhibitor to the subject.

11. The method of claim 10, further comprising administering a receptor tyrosine kinase inhibitor to the subject.

12. The method of claim 1 1, wherein the receptor tyrosine kinase inhibitor is a c-Kit inhibitor.

13. A method for identifying an agent which can be used to treat small cell lung cancer comprising determining whether the agent inhibits CXCR4.

14. A method for determining whether a CXCR4 inhibitor can or cannot be used to treat small cell lung cancer comprising:
a) obtaining a sample of lung cancer cells; and
b) determining whether the cells express CXCR4;
thereby determining that the CXCR4 inhibitor can be used to treat small cell lung cancer when CXCR4 is expressed.

15. A method for determining whether a combination of a CXCR4 inhibitor and a receptor tyrosine kinase inhibitor can or cannot be used to treat small cell lung cancer comprising:
a) obtaining a sample of lung cancer cells; and
b) determining whether the cells express CXCR4 and the receptor tyrosine kinase that is inhibited by said receptor tyrosine kinase inhibitor;
thereby determining that the combination of a CXCR4 inhibitor and a receptor tyrosine kinase inhibitor can be used to treat small cell lung cancer when CXCR4 and the receptor tyrosine kinase are expressed.

16. The method of claim 15, wherein the receptor tyrosine kinase inhibitor is a c-Kit inhibitor.

17. A method for determining whether a CXCR4 inhibitor can or cannot be used to inhibit proliferation or metastasis of small cell lung cancer comprising:
a) obtaining a sample of lung cells; and
b) determining whether the cells express CXCR4;
thereby determining that the CXCR4 inhibitor can be used to inhibit proliferation or metastasis of small cell lung cancer when CXCR4 is expressed.

18. A method for determining whether a combination of a CXCR4 inhibitor and a receptor tyrosine kinase inhibitor can or cannot be used to inhibit proliferation or metastasis of small cell lung cancer comprising:
a) obtaining a sample of lung cells; and
b) determining whether the cells express CXCR4 and the receptor tyrosine kinase that is inhibited by said receptor tyrosine kinase inhibitor;
thereby determining that the combination of a CXCR4 inhibitor and a receptor tyrosine kinase inhibitor can be used to inhibit proliferation or metastasis of small cell lung cancer when CXCR4 and the receptor tyrosine kinase are expressed.

19. The method of claim 18, wherein the receptor tyrosine kinase inhibitor is c-Kit inhibitor.

20. A method for determining whether a patient would benefit from treatment with an agent that inhibits CXCR4 comprising:
a) obtaining a lung sample from the patient; and
b) determining whether CXCR4 is expressed in the sample.

21. A method for determining whether a patient would benefit from treatment with an agent that inhibits CXCR4 and an agent that inhibits c-Kit comprising:
a) obtaining a lung sample from the patient;
b) determining whether CXCR4 is expressed in the sample; and
c) determining whether c-Kit is expressed in the sample.

22. A method for determining whether treatment with a CXCR4 inhibitor should be continued or discontinued in a small cell lung cancer patient, comprising:
a) obtaining two or more samples comprising tumor cells from a patient during the course of treatment;
b) determining the level of activity in the tumor cells of CXCR4; and
c) continuing treatment when the activity of CXCR4 does not increase during treatment.

23. A method for determining whether treatment with a combination of a CXCR4 inhibitor and a receptor tyrosine kinase inhibitor should be continued or discontinued in a small cell lung cancer patient, comprising:
a) obtaining two or more samples comprising tumor cells from a patient during the course of treatment;
b) determining the level of activity in the tumor cells of CXCR4;
c) determining the level of activity in the tumor cells of c-Kit; and
d) continuing treatment when the activity levels of CXCR4 and/or c-Kit do not increase during treatment.

24. A CXCR4 inhibitor for use in a method for the treatment of a subject.

25. Use of a CXCR4 inhibitor for the preparation of a pharmaceutical composition for use in a method such as inhibiting cellular proliferation in a small cell lung cancer cell population, inhibiting cellular movement or motility in a small cell lung cancer cell population, modulating cellular adhesion in a small cell lung cancer cell population, inhibiting morphological change in a small cell lung cancer cell population, treating a subject having small cell lung cancer, or inhibiting metastasis of small cell lung cancer in a subject.

26. A combination which comprises (a) a CXCR4 inhibitor and (b) a receptor tyrosine kinase inhibitor, such as especially a c-Kit inhibitor, wherein the active ingredients (a) and (b) are present in each case in free form or in the form of a pharmaceutically acceptable salt, for simultaneous, concurrent, separate or sequential use, especially in a method for the treatment of a subject, preferably in a method such as inhibiting cellular proliferation in a small cell lung cancer cell population, inhibiting cellular movement or motility in a small cell lung cancer cell population, modulating cellular adhesion in a small cell lung cancer cell population, inhibiting morphological change in a small cell lung cancer cell population, treating small cell lung cancer, or inhibiting metastasis of small cell lung cancer.
